(19) United States
(12) Patent Application Publication
(10) Pub. No.: US 2026/0355473 A1
(43) Pub. Date: Nov. 12, 2026

(54) GUIDE EXTENSION CATHETER

(71) Applicants: Boston Scientific Scimed, Inc., Maple Grove, MN (US); Boston Scientific Medical Device Limited, Galway (IE)

(72) Inventors: Shubham Gupta, Gurugram (IN); Jeffrey Steven Fuller, Brooklyn Park, MN (US); Sumit Malik, Gurgaon (IN); Shinto Sebastian, Malappuram (IN); Neeraj Kumar Sharma, New Delhi (IN)

(73) Assignees: Boston Scientific Scimed, Inc., Maple Grove, MN (US); Boston Scientific Medical Device Limited, Galway (IE)

(21) Appl. No.: 19/675,082

(22) Filed: May 12, 2026

Related U.S. Application Data
(60) Provisional application No. 63/804,130, filed on May 12, 2025.

(51) Int. Cl. A61M 25/06; A61M 29/00; A61B 17/34

(52) U.S. Cl. CPC A61M 25/0662; A61M 29/00; A61B 17/3496; A61M 2025/0681; A61M 2210/12

(57) ABSTRACT

Medical devices and methods for making and using medical devices and systems are disclosed. An example medical system may include a guide extension catheter and a dilator device. The dilator device may be configured to be inserted within the guide extension catheter. The guide extension catheter may include a proximal end, a distal end, a guide extension hub at a proximal end and a guide extension tube at the distal end. The dilator device may include a dilator pusher, a dilator tip defining a distal end of the dilator device, and a distal stopper at a location proximal of the dilator tip and coupled with the dilator pusher. The guide extension hub may be configured to couple with the dilator pusher.

### CROSS-REFERENCE TO RELATED APPLICATIONS

This application claims the benefit of U.S. Provisional Patent Application Serial No. 63/804,130 filed on May 12, 2025, the disclosure of which is incorporated herein by reference.

### TECHNICAL FIELD

The present disclosure pertains to medical devices, and methods for manufacturing medical devices. More particularly, the present disclosure pertains to elongate intracorporeal medical devices including a guide extension catheter

### BACKGROUND

A wide variety of intracorporeal medical devices have been developed for medical use, and more specifically for intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. In one example, a guide extension catheter system may include a guide extension catheter having a proximal end, a distal end, a guide extension hub at a proximal end, and a guide extension tube at the distal end and a dilator configured to be inserted within the guide extension tube. The dilator may include a dilator pusher, a dilator tip defining a distal end of the dilator, and a distal stopper at a location proximal of the dilator tip and coupled with the dilator pusher, wherein the guide extension hub may be configured to couple with the dilator pusher.

Additionally or alternatively to any of the examples above, the guide extension hub of the guide extension catheter may include a connector configured to receive and engage the dilator pusher.

Additionally or alternatively to any of the examples above, the connector may be configured to axially fix the dilator relative to the guide extension catheter.

Additionally or alternatively to any of the examples above, the distal stopper may be configured to engage the guide extension tube.

Additionally or alternatively to any of the examples above, the dilator tip may be configured to extend from a distal end of the guide extension tube to a location distal of the distal end of the guide extension tube when the distal stopper is engaged with the guide extension tube and the guide extension hub is coupled with the dilator pusher.

Additionally or alternatively to any of the examples above, the system may include a carrier tube configured to extend over the guide extension hub and the dilator pusher.

Additionally or alternatively to any of the examples above, the guide extension hub may include a connector configured to engage the dilator pusher and the carrier tube is configured to extend over the connector when the connector is engaging the dilator pusher.

Additionally or alternatively to any of the examples above, the guide extension catheter may include a guide extension pusher extending between the guide extension hub and the guide extension tube, and the carrier tube is configured to extend over the guide extension pusher, the guide extension hub, and the dilator pusher.

Additionally or alternatively to any of the examples above, the dilator may include a dilator hub coupled with a proximal end of the dilator pusher.

Additionally or alternatively to any of the examples above, the system may include a connector, wherein the guide extension hub may be configured to couple with the dilator pusher via the connector.

Additionally or alternatively to any of the examples above, the connector may be configured to create a clip connection with the dilator hub and the guide extension hub.

In an example, a method of using a guide extension catheter system may include inserting a dilator tip of a dilator through a tube of a guide extension catheter, wherein the guide extension catheter comprises the tube and a guide extension hub and the dilator comprises the dilator tip and a dilator pusher, inserting the dilator into a connector to couple the dilator with the guide extension catheter, and delivering the dilator to a target location through vasculature of a patient while the dilator is coupled with the guide extension catheter using the connector.

Additionally or alternatively to any of the examples above, the method may include engaging a dilator stopper with the tube of the guide extension catheter.

Additionally or alternatively to any of the examples above, the connector may be part of the guide extension hub and inserting the dilator into the connector to couple the dilator with the guide extension catheter comprises inserting the dilator pusher into the connector.

Additionally or alternatively to any of the examples above, inserting the dilator into the connector to couple the dilator with the guide extension catheter may include connecting a dilator hub coupled to the dilator pusher with the connector and connecting the guide extension hub with the connector.

Additionally or alternatively to any of the examples above, the method may include removing the dilator tip from the tube of the guide extension catheter and delivering a treatment device through the tube of the guide extension catheter to the target location.

Additionally or alternatively to any of the examples above, delivering the dilator to the target location may include delivering the dilator to the target location over a guidewire extending through the dilator tip and a dilator stopper of the dilator.

In an example, a guide extension catheter system may include a guide extension catheter having a proximal end, a distal end, a guide extension hub at a proximal end, and a guide extension tube at the distal end and a dilator configured to be inserted within the guide extension tube. The dilator may include a dilator pusher, a dilator tip defining a distal end of the dilator, and a distal stopper at a location proximal of the dilator tip and coupled with the dilator pusher, wherein the guide extension hub may include a clip configured to receive and engage the dilator pusher.

Additionally or alternatively to any of the examples above, the guide extension hub may include a handle portion proximal of the clip.

Additionally or alternatively to any of the examples above, the method may include a carrier tube configured to extend over the clip and the dilator pusher while the clip is engaged with the dilator pusher.

The above summary of some configurations is not intended to describe each disclosed configuration or every implementation of the present invention. The Figures, and Detailed Description, which follow, more particularly illustrate these configurations.

### BRIEF DESCRIPTION OF THE DRAWINGS

The invention may be more completely understood in consideration of the following detailed description of various configurations of the invention in connection with the accompanying drawings, in which:

FIG. 1 depicts a schematic plan view of an illustrative guide catheter advanced through the aorta to the ostium of a coronary artery;

FIG. 2 depicts a schematic plan view of an illustrative guide extension catheter used in conjunction with a guide catheter;

FIG. 3 depicts a schematic cross-sectional view of an illustrative guide extension catheter;

FIG. 4 depicts a schematic cross-sectional view of the illustrative guide extension catheter and an illustrative guide catheter;

FIG. 5 depicts a schematic exploded view an illustrative guide extension catheter system;

FIG. 6 depicts a schematic side view of the illustrative guide extension catheter system depicted in FIG. 5;

FIG. 7 depicts a schematic cross-section view of a distal portion of an illustrative guide extension catheter system;

FIG. 8 depicts a schematic side view of a distal portion of an illustrative guide extension catheter system;

FIG. 9 depicts a schematic cross-section view of the illustrative guide extension catheter system depicted in FIG. 8, taken along line 9-9;

FIG. 10 depicts a schematic cross-section view of the illustrative guide extension catheter system depicted in FIG. 8, taken along line 10-10;

FIG. 11 depicts a schematic perspective view of a proximal portion of an illustrative guide extension catheter system;

FIGS. 12A and 12B depict a schematic perspective view and a schematic cross-section view, respectively, of a proximal portion of an illustrative guide extension catheter system;

FIG. 13 depicts a schematic perspective view of a proximal portion of an illustrative guide extension catheter system;

FIG. 14 depicts a schematic diagram of an illustrative method of using a guide extension catheter system;

FIG. 15 depicts a schematic diagram of an illustrative method of using a guide extension catheter system; and

FIGS. 16-23 depict schematic diagrams of steps of an illustrative method of using a guide extension catheter system.

While the disclosure is amenable to various modifications and alternative forms, specifics thereof have been shown by way of example in the drawings and will be described in detail. It should be understood, however, that the intention is not to limit the invention to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar elements in different drawings are numbered the same. The drawings, which are not necessarily to scale, depict illustrative configurations and are not intended to limit the scope of the invention.

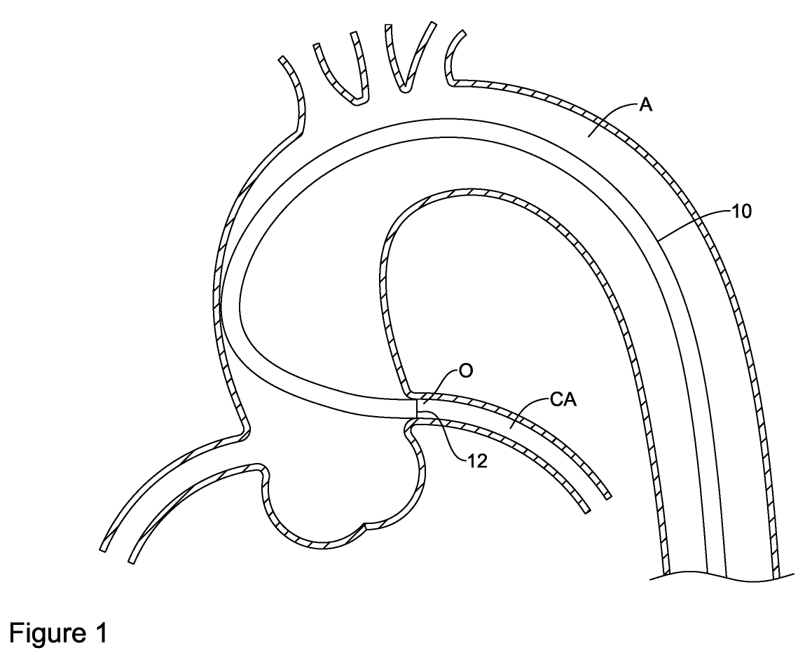

Minimally-invasive vascular interventions (e.g., cardiac interventions such as percutaneous transluminal coronary angioplasty) are widely utilized throughout the world. These procedures may include the use of a guide catheter. For example, a guide catheter 10 may be advanced through a blood vessel, such as an aorta A to a position adjacent to an ostium O of a (e.g., left and/or right) coronary artery CA as illustrated in FIG. 1. When so positioned, an ancillary device (e.g., a treatment device, such as a balloon catheter, a stent delivery system, etc.) may be advanced through guide catheter 10 and into the coronary artery CA to a target location where the ancillary device may be used to perform the appropriate cardiac intervention or other suitable intervention in the blood vessel.

In order for the ancillary device to efficiently reach the intended target location, maintaining the position of guide catheter 10 at the ostium O of the coronary artery CA or other suitable location may be desirable. In one example, given that the heart may be beating during the intervention (and/or other factors), the guide catheter 10 may lose its positioning or otherwise be shifted so that it no longer is positioned to efficiently guide the ancillary device to the coronary arteries. This may include a distal end 12 of guide catheter 10 being shifted away from the ostium O of the coronary artery CA. Because of the shift away from the ostium O, access to the coronary arteries CA may require repositioning of guide catheter 10 in order to bring the distal end 12 back into engagement with the ostium O of the coronary artery CA. In addition to facilitating the maintenance of the guide catheter 10 at the ostium O of the coronary artery CA, it may be desirable to advance a catheter to a location closer to the target location in a vessel to facilitate delivering the ancillary device to the target location.

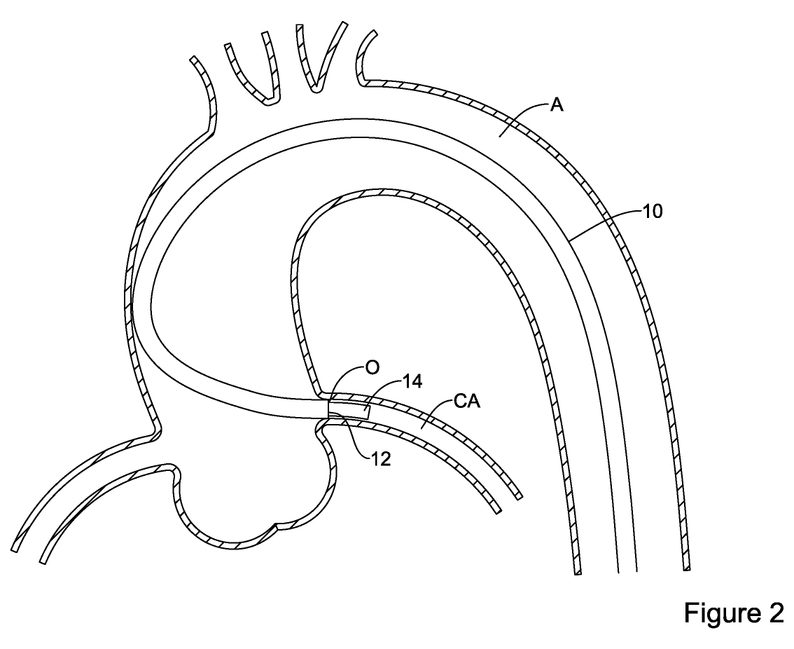

Disclosed herein are medical devices, systems, and methods for making and using medical devices and systems that may improve access to the coronary arteries CA and/or other vasculature (e.g., blood vessels) throughout a body of a patient. An example of such a medical device is depicted in FIG. 2, which illustrates a guide extension catheter 14 extending through the guide catheter 10 and beyond the distal end 12 of the guide catheter 10 into the coronary artery CA. Because, for example, the guide extension catheter 14 may extend beyond the distal end 12 of the guide catheter 10, the guide extension catheter 14 may extend beyond the ostium O of the coronary artery CA and into a portion of the coronary artery CA. By extending beyond the ostium O, the guide extension catheter 14 may stabilize the positioning of the guide catheter 10 and allow for improved access to the coronary artery CA for a number of cardiac interventions. The guide catheter 10 and the guide extension catheter 14 may be used in a similar or different manner in other vessels.

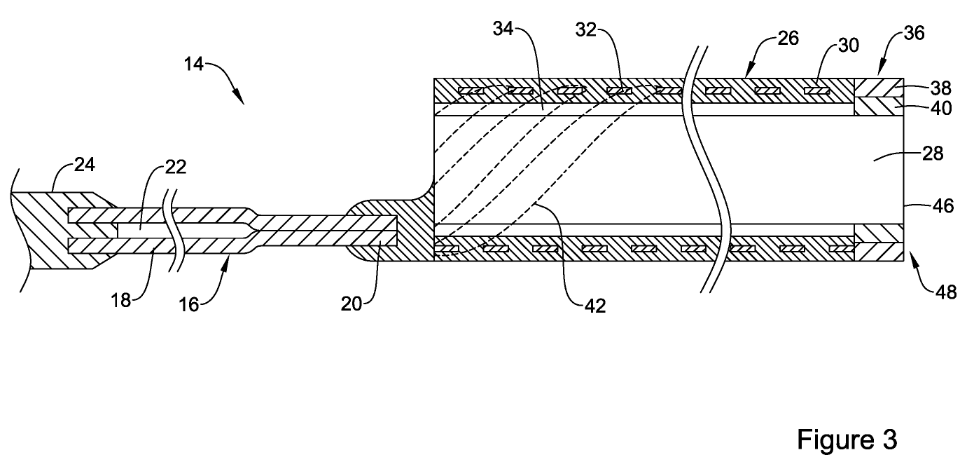

FIG. 3 is a cross-section view of the guide extension catheter 14. The guide extension catheter 14 may include a proximal shaft or member 16. The member 16 may include a proximal portion 18 and a distal or ribbon portion 20. The proximal portion 18 may have one or more lumens 22 defined therein, but other suitable configurations without the lumen(s) 22 are contemplated. In some configurations, the lumen 22 may extend along an entire length of the proximal portion 18. In other configurations, the lumen 22 may extend along only a portion of the length of the proximal portion 18. In still other configurations, the member 16 may be a hypotube and have a lumen extending along the entire length of the member 16. In addition, the proximal portion 18 may include both proximal and distal openings (e.g., positioned at the proximal end and the distal end of the proximal portion 18) such that the lumen 22 is “open” on both ends. Alternatively, one or both of the ends of the proximal portion 18 may be closed or otherwise sealed. For example, the distal end of the proximal portion 18 may be closed. In some of these and in other configurations, the proximal portion 18 may have an opening or port (not shown) formed in the wall of the proximal portion 18 and spaced from the proximal end and/or the distal end of the proximal portion 18. The port may or may not be in fluid communication with the lumen 22. A handle or hub 24 may be attached to the proximal portion 18.

A tube 26 (e.g., a distal tube, a guide extension sheath, a distal sheath, etc.) may be attached to the member 16. The tube 26 may have a lumen 28 formed therein. In general, the lumen 28 (and/or the inner diameter of the tube 26) may be larger than the lumen 22 (and/or the inner diameter of proximal portion 18) and may be larger than the outer diameter of the member 16. As used herein, the term diameter may refer to a line from one side of something to a second side of the something, wherein the line passes through a center point of the something and/or the widest portion of the something. Illustratively, the something may be a cross-sectional shape of a square, a circle, a triangle, an oval, a rectangle, or other shape. With respect to the proximal member 16 and/or the tube 26 that may or may not have a circular cross-sectional shape, the diameter of such shapes may be the largest cross-sectional dimension of the proximal member 16 or the tube 26 taken from a cross-section transverse to a longitudinal dimension (e.g., to a direction of a longitudinal axis). Accordingly, the lumen 28 may be sufficiently large so as to allow a therapeutic catheter (e.g., a balloon catheter, a stent delivery system, etc.) to pass therethrough. For example, when the guide extension catheter 14 is positioned within the guide catheter 10, the therapeutic catheter may extend within the guide catheter 10 alongside member 16 and through the lumen 28 of the tube 26.

The tube 26 may include a body portion 30. In at least some configurations, the body portion 30 may include one or more polymers including any of those disclosed herein. This may include the use of polymers with a differing durometer along the length of the body portion 30. For example, a more proximal section of the body portion 30 may include a polymer with a higher durometer and a more distal section of the body portion 30 may include a polymer with a lower durometer. Portions of all of the length of the body portion 30 may be loaded with or otherwise include a radiopaque material. The body portion 30 may also include a reinforcement member 32. The form of the reinforcement member 32 may vary. For example, the reinforcement member 32 may include a braid, coil, mesh, rings, segments, fibers or interstitial arrays of fibers, or the like.

An inner layer or liner 34 may be disposed along an inner surface of the body portion 30. The form of the liner 34 may vary. For example, the liner 34 may be a lubricious liner or otherwise include a lubricious material such as polytetrafluoroethylene (PTFE) and/or other suitable lubricious material. A tip member 36 may be attached to the body portion 30, for example at a distal end of body portion 30. In some configurations, the tip member 36 may be a single layer of material. Alternatively, the tip member 36 may include an outer layer 38 and an inner layer 40. The outer layer 38 and the inner layer 40 may be formed from the same material. In some configurations, the outer layer 38 and the inner layer 40 may include the same polymeric material and each may be loaded with the same or different radiopaque materials. For example, the inner layer 40 may include a polyether block amide (PEBA) loaded with approximately 75-95% (e.g., about 90%) by weight tungsten and the outer layer 38 may include a PEBA loaded with approximately 30-50% (e.g., 40%) by weight bismuth subcarbonate. These are just examples. In other configurations, the outer layer 38 and the inner layer 40 may be made from different materials.

The tube 26 may be attached to the ribbon portion 20 of the member 16. The arrangement and/or configuration of the attachment between ribbon portion 20 and tube 26 may vary. For example, tube 26 may have an opening or lumen formed in a tube wall thereof and the ribbon portion 20 may be disposed within the opening. This may include necking, skiving, or pinching down the ribbon portion 20 and inserting the necked down portion into the opening. In some configurations, inserting the ribbon portion 20 into the opening may secure the member 16 to the tube 26 via a mechanical bond. In some and in other configurations, additional and/or alternative bonding may be utilized including those bonding mechanisms commonly used for medical devices. Some example bonding mechanisms include adhesive bonding, welding, thermal bonding, brazing, metallurgical stamping, pressing etc. Other attachment mechanisms are also contemplated for attaching the member 16 to the tube 26 including direct bonding (e.g., adhesive bonding, thermal bonding, welding, brazing, injection molding, 3D printing and bonding, overmolding, casting, sintering polymer/metal composites, etc.), bonding that is facilitated by a third component such as a metal or polymer collar 42 that may be bonded between and/or otherwise coupled with the ribbon portion 20 and the tube 26, where the tube 26 may extend in a distal direction from the collar 42 and the ribbon portion 20 may extend in a proximal direction from the collar 42. In some examples, the tube 26 coupled with collar 42 may form a guide extension tube of the guide extension catheter 14.

The guide extension catheter 14 may also include a number of coatings that may, for example, reduce friction. For example, the proximal member 16 may have an inner and/or outer coating that includes a hydrophilic polymer that may reduce friction during tracking. An example coating may include BAYER CL-100, BIOSLIDE, NG-HPC, SLIP COAT, MDX, ZGLIDE, or the like. These are just examples. Other suitable materials are contemplated including those disclosed herein.

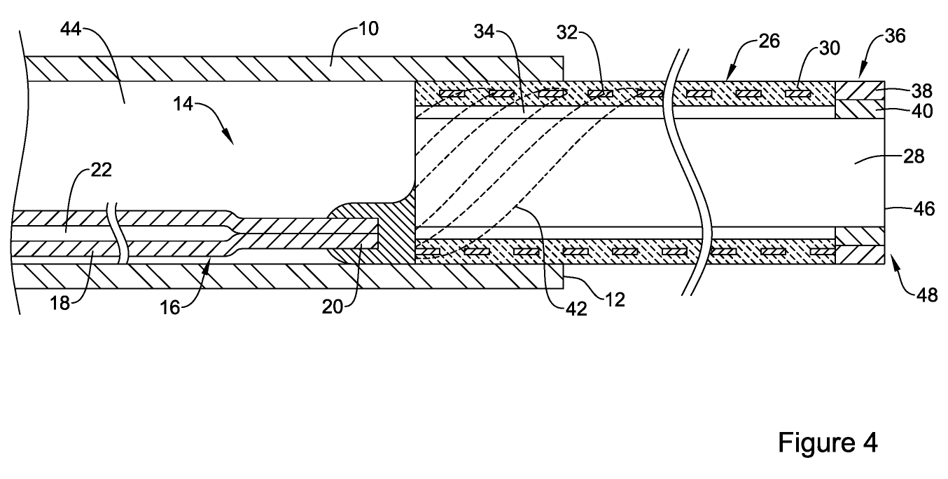

FIG. 4 illustrates the guide extension catheter 14 disposed within the guide catheter 10 (e.g., disposed within a lumen 44 defined within the guide catheter 10). As shown, the tube 26 may be arranged to extend distally out from the distal end 12 of the guide catheter 10. When so arranged, the tube 26 may engage the ostium O and/or extend within a portion of the coronary artery CA or other suitable vessel to help maintain the position of the guide catheter 10 and improve access to the coronary artery CA and/or other suitable vessel. The member 16 may be designed to be sufficiently small (while still being sufficiently sized and configured for pushability) so as to take up relatively little space within the interior or lumen 44 of guide catheter 10. Accordingly, the use of the guide extension catheter 14 allows for a therapeutic catheter or medical device to be advanced through the guide catheter 10 in order to reach the desired target location for the intervention. In some examples, the member 16 may contact the inner wall surface of the guide catheter 10, which may provide even more space.

As discussed, some configurations of the guide extension catheter 14 may include the collar 42 connected between the member 16 and the tube 26, as illustrated in FIGS. 3 and 4. In some configurations, the collar 42 may be sized to work with configurations of the guide catheter 10 or the guide extension catheter 14 that have sizes between about 4 French to about 9 French. Additionally or alternatively, the collar 42 may be sized differently to cooperate with the guide catheters 10 and/or the guide extension catheters 14 that are smaller than 4 French and larger than 9 French.

A distal opening 46 at a distal end 48 of the guide extension catheter 14 may be larger than a guidewire on which the guide extension catheter 14 tracks. Similarly, an outer diameter of the guide extension catheter 14 at the distal end 48 may also be larger than the guidewire on which the guide extension catheter 14 tracks. As such, the distal end of 48 of the guide extension catheter 14 may present a catch risk within a vessel in which the guide extension catheter 14 is advanced. Catching of the guide extension catheter 14 on the vessel may result in a dissection of the vessel and/or other damage to the vasculature of the patient.

To facilitate advancing the guide extension catheter 14 to a target location in the vasculature for deploying an ancillary device and performing a treatment with the ancillary device, one or more devices or components may be utilized to assist with the advancement of the guide extension catheter 14 to the target location within the vessel and distal of the guide catheter 10. In some examples, a balloon (e.g., via balloon assisted tracking (BAT)) may be used to assist in advancing the guide extension catheter 14 to the target location while mitigating the catch risk associated with advancing the guide extension catheter through a vessel. Example BAT techniques include, but are not limited to, using a balloon with a rail technique, using a balloon with a tip technique, using an “inchworm” technique in which a balloon is inflated and deflated to facilitate advancing the guide extension catheter 14 incrementally, using a balloon with an anchor technique in which the guide extension catheter 14 may be pushed in a distal direction while the balloon may be pulled proximally, and/or other suitable techniques for advancing the guide extension catheter 14 to the target location within a vessel in a manner that prevents or mitigates a likelihood of the distal end 48 of the guide extension catheter 14 catching on the vessel.

BAT techniques for delivering guide extension catheters or other suitable catheters to a target location in a vessel may be undesirable for one or more reasons. For example, BAT techniques require use of a balloon catheter for tracking or delivery of the guide extension catheter 14, are time consuming and can add extra steps to a procedure, and/or do not eliminate all likelihood of catching a vessel with the distal end 48 of the guide extension catheter 14 such that users may lack confidence in using BAT techniques.

In addition to or as an alternative to using a balloon catheter with the guide extension catheter 14 to advance the guide extension catheter 14 to the target location within the vessel, a dilator device may be used with the guide extension catheter 14 to create a guide extension catheter system and facilitate delivering the guide extension catheter 14 to the target location within the vessel. In some examples, a dilator tip of the dilator device may extend distal of the distal end of the guide extension catheter and have a proximal outer diameter that is the same as or close to the outer diameter of the distal end 48 of the guide extension catheter 14 to prevent or mitigate a likelihood of the guide extension catheter 14 catching on or injuring the vessel during advancement of the guide extension catheter 14. Additionally, the dilator tip may be configured to center and/or direct the guide extension catheter 14 to follow the guide wire extending to the target location. The stopper may engage the collar 42 and/or proximal portion of the tube 26 of the guide extension catheter 14, which may maintain the dilator tip at a desired location relative to the distal end 48 of the guide extension catheter 14 and facilitate pushing the guide extension catheter 14 to the desired location.

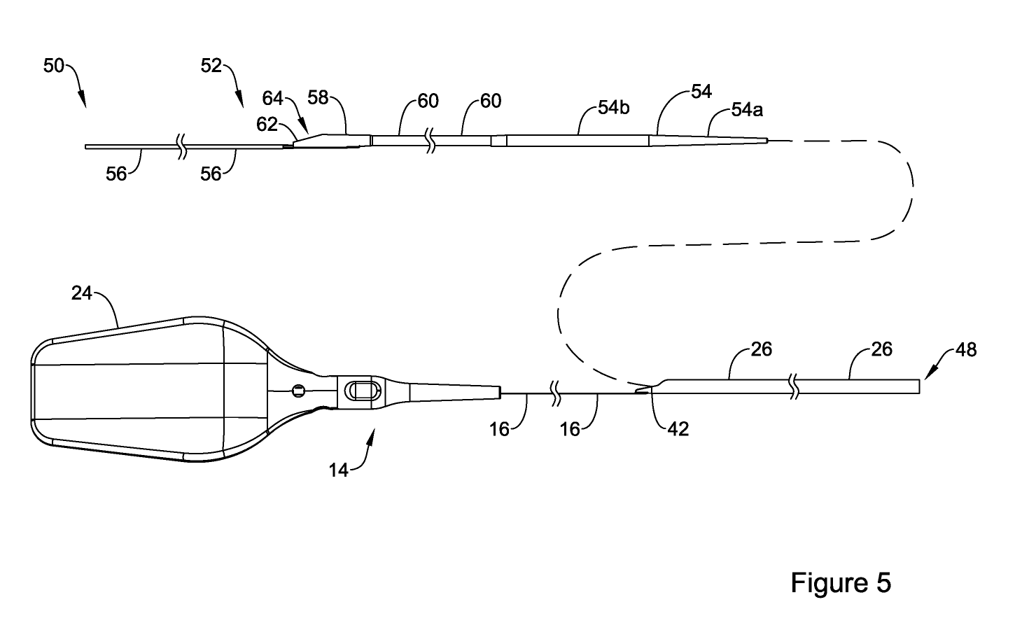

FIG. 5 depicts a schematic exploded view of an illustrative guide extension catheter system 50 including the guide extension catheter 14 with the handle or hub 24 (e.g., a guide extension hub) and a dilator device 52 configured to facilitate delivery of the guide extension catheter 14 to a location at or proximate a target location within a vessel. The dilator device 52 may be configured to be inserted within the guide extension catheter 14 and center and/or direct the guide extension catheter 14 to follow a guidewire extending to the target location or other suitable desired location within a vessel. In some examples, the dilator device 52 may be configured to engage the guide extension catheter 14 such that a desired length of the dilator device 52 extends distally from the distal end 48 of the guide extension catheter 14, which may facilitate pushing and advancing the guide extension catheter 14 to the target location.

The dilator device 52 may have any suitable configuration. In some examples, the dilator device 52 may include a dilator tip 54 (e.g., a distal tip) defining a distal end of the dilator device 52 and a proximal member 56, with the proximal member 56 coupled with the dilator tip 54 and extending in a proximal direction from the dilator tip 54 to allow a user to manipulate the dilator device 52 longitudinally and/or rotationally relative to and/or with the guide extension catheter 14. In some examples, the dilator device 52 may include additional or alternative components including, but not limited to, a stopper 58 (e.g., a distal stopper) or other suitable locking mechanism, a tube 60 defining a guidewire lumen, and/or other suitable components.

The components of the dilator device 52 may have any suitable arrangement relative to one another. In some examples, the proximal member 56 may be coupled with the stopper 58, the stopper 58 may be proximal of the dilator tip 54, the tube 60 may be coupled to the stopper 58 and extend to the dilator tip 54. In some examples, the tube 60 may be coupled to the dilator tip 54. Although other suitable configurations are contemplated, the tube 60 may at least partially define a guidewire port 64 at a proximal end of the stopper 58 and a guidewire lumen may extend from the guidewire port 64 to the dilator tip 54.

The proximal member 56 of the dilator device 52 may have any suitable configuration. In some examples, the proximal member 56 may be configured as a push wire, a proximal pusher, dilator pusher, and/or other suitable member extending proximally from the stopper 58. In some examples, the proximal member 56 may have a solid cross-section and/or may have a tubular cross-section defining a lumen. The cross-section of the proximal member 56 may be consistent (e.g., the same shape) along an entire length of the proximal member 56 or may have different configurations along sections or portions of the length of the proximal member 56. In some examples, when the proximal member 56 includes a lumen, the lumen may extend along the entire length of the proximal member 56. In other examples, the lumen may extend along only one or more portions of the length of proximal member 56. In one example, the proximal member 56 may be a hypotube and have a lumen extending along the entire length of the proximal member 56. In one example, the proximal member 56 may be a wire or ribbon without a lumen. When the proximal member 56 includes a lumen, the proximal member 56 may include both a proximal opening and a distal opening (e.g., positioned, respectively, at the proximal end and the distal end of the proximal member 56) such that the lumen is “open” on both ends. Alternatively, one or both of the proximal end and the distal end of the proximal member 56 may be closed or otherwise sealed. For example, the distal end of the proximal member 56 may be closed. In some of these configurations and/or in other configurations, the proximal member 56 may have an opening or port (not shown) formed in the wall of the proximal member 56 and spaced from the proximal end and/or the distal end of the proximal member 56. The port may or may not be in fluid communication with the lumen, when the lumen is included in the proximal member 56. Although not depicted in FIG. 5, a handle or hub may be attached to the proximal end of the proximal member 56.

The proximal member 56 may be designed to have a sufficiently small cross-sectional dimension, while still being configured for pushability, so as to have a desired flexibility and take up relatively little space within the interior of the lumen 44 of the guide catheter 10. In some examples, the proximal member 56 may contact the inner wall surface of the guide catheter 10, which may provide even more space in the lumen 44. Although other outer diameters of the proximal member 56 are contemplated, the proximal member 56 may have an outer diameter in a range of about 0.254 millimeters (mm) (0.010 inches) to about 0.610 mm (0.024 inches). In one example, the proximal member 56 may have an outer diameter of 0.356 mm (0.014 inches) at a proximal end of the stopper 58. In one example, the proximal member 56 may have an outer diameter that tapers from about 0.457 mm (0.018 inches) at a proximal end of the proximal member 56 to about 0.254 millimeters (mm) (0.010 inches) at the distal end of the proximal member 56, with the proximal member 56 having an outer diameter of about 0.356 mm (0.014 inches) at region along the stopper 58.

The proximal member 56 may be configured to extend from a proximal end to the stopper 58 and/or beyond the stopper 58. In some examples, the proximal member 56 may extend to the dilator tip 54, but other suitable configurations are contemplated. When the proximal member 56 extends to the dilator tip 54, the proximal member 56 may be coupled to the stopper and/or the dilator tip 54. In some examples, the proximal member 56 may extend to the stopper 58 to facilitate coupling with the stopper 58, but not extend beyond the stopper 58.

A distal end of or other suitable portions of the proximal member 56 may be configured to couple with the stopper 58. For example, the distal end or other suitable portion of the proximal member 56 may have a flattened area, a flattened arcuate area, a stump area, an area with a textured surface or holes, and/or other suitable configuration that may facilitate coupling the proximal member 56 with the stopper 58.

The stopper 58 may have any suitable configuration configured to engage or couple with the tube 26 and/or the collar 42. For example, the stopper 58 may be configured to engage the tube 26 and/or the collar 42 with a friction fit, an interference fit, a bayonet fit, a threaded fit, and/or other suitable reversible coupling technique. In one example, the stopper 58 may be configured to engage the tube 26 and/or the collar 42 of the guide extension catheter with a friction fit. In some examples, a cross-section of the stopper 58 may have a circular shape or other suitable shape configured to mate with an inner diameter of the tube 26 and/or the collar 42 and facilitate coupling the stopper 58 with the tube 26 and/or the collar 42.

The stopper 58 may have any suitable cross-sectional dimensions at one or more axial locations. In some examples, the stopper 58 may have an outer diameter greater than an outer diameter of the proximal member 56 and the outer diameter of at least a portion of the stopper 58 may be greater than an inner diameter of the tube 26 and/or the collar 42. In some examples, outer diameter of the stopper 58 may be sized to create an interference fit with the tube 26 and/or the collar 42. In one example, the outer diameter of the stopper 58 may have one or more tapers, where the taper may be configured such that the stopper 58 engages and creates a friction fit with the tube 26 and/or the collar 42 at a known or predetermined location to ensure the dilator tip 54 extends a known or predetermined distance beyond a distal end of the tube 26 (e.g., beyond the tip member 36).

The stopper 58 may include a proximal taper or bevel 62. In some examples, the proximal bevel 62 of the stopper 58 may include or define the guidewire port 64 configured to receive a guidewire into and/or through an opening of or lumen through the stopper 58. The guidewire port 64 may be or be configured as a rapid exchange port, but other suitable configurations are contemplated. When the guidewire port 64 is formed as a rapid exchange port, the guidewire port 64 may facilitate using a short, rapid exchange guidewire (e.g., approximately 260 centimeters (cm)), rather than a long, over-the-wire guidewire (e.g., approximately 450 cm). The guidewire port 64 may be configured to receive a guidewire having any suitable diameter including, but not limited to, 0.356 mm (0.014 inches), about which the dilator device 52 may travel as the components of the guide extension catheter system 50 travel to and/or from the target location.

The stopper 58 may be formed from one or more suitable biocompatible materials. In some examples, the stopper 58 may be formed from a resilient material configured to engage the tube 26 and/or the collar 42 to create an interference fit at a known location. Example suitable materials for forming the stopper 58 include, but are not limited to, the biocompatible materials discussed herein, metals, polymers, silicone, thermoplastic elastomers (TPEs), a block copolymer, nylon materials (e.g., Nylon 11, Nylon 12, TR55, etc.), acrylonitrile butadiene styrene (ABS), polycarbonate, PEBA (e.g., PEBAX®), PTFE, and/or other suitable materials. In one example, the stopper 58 may be formed from PEBA. In one example, the stopper 58 may be formed from ABS. In one example, the stopper 58 may be formed from Nylon 11. In one example, the stopper 58 may be formed from Nylon 12. In some examples, the stopper 58 may be formed from a single material in a single layer of material, a single material in a plurality of layers of materials, or a plurality of materials in a plurality of layers.

The tube 60 (e.g., an elongate tube) may have any suitable configuration configured to receive a guidewire extending through the guidewire port 64. In some examples, a proximal end of the tube 60 may at least partially define the guidewire port 64. The tube 60 may define a lumen configured to receive the guidewire along which the dilator device 52 may track as the dilator device 52 travels to a target location in a vessel. In some examples, the proximal member 56 may form the tube 60, but other suitable configurations are contemplated. In some examples, the tube 60 may have a length sufficient to extend between the stopper 58 and the dilator tip 54. In one example, the tube 60 may extend from a proximal portion of the stopper 58 to a proximal portion of the dilator tip 54 with which the tube 60 may be coupled. In some examples, the tube 60 may extend to the distal end of the dilator tip 54, but other suitable configurations are contemplated. When the tube 60 extends from a proximal end of the stopper 58, the proximal end of the tube 60 may be tapered or beveled at a same or similar angle to an angle of the taper or bevel 62 of the stopper 58 to facilitate receiving the guidewire through a proximal opening (e.g., at the guidewire port 64).

The tube 60 may be any suitable type of tube formed from any suitable type of material. For example, the tube may be a catheter, a sleeve, a hypotube, and/or suitable type of tube. Example suitable types of materials of which the hypotube may be formed include, but are not limited to, metals, polymers, fabrics, silicone, thermoplastic elastomers, and/or other suitable materials. In one example, the tube 60 may be a sleeve formed from a polymer. Additionally or alternatively, one or more coatings may be applied to an inner surface and/or outer surface of the tube 60. When applied to the tube, the coatings may be part of or separate from the tube 60

The tube 60 may have any suitable length configured to extend between stopper 58 and the dilator tip 54. In some examples, tube 60 may have a length configured to facilitate extending the dilator tip 54 beyond the distal end of the guide extension catheter 14 a known or predetermined distance when the stopper 58 is coupled with the tube 26 and/or the collar 42. Example suitable lengths of the tube 60 include, but are not limited to, a length in a range of about 20 centimeters (cm) (7.874 inches) to about 40 cm (15.748 inches), a length in a range of about 20 centimeters (cm) (7.874 inches) to about 24 cm (9.448 inches), a length in a range of about 35 cm (13.780 inches) to about 39 cm (15.354 inches), and/or other suitable lengths. Other suitable lengths of the tube 60 of the dilator device 52 are contemplated.

The dilator tip 54 of the dilator device 52 may have any suitable configuration that is configured to extend within and/or beyond a distal end of the guide extension catheter 14 when the distal stopper 58 is engaged with the guide extension catheter 14. The dilator tip 54 may facilitate delivering the guide extension catheter system 50 to the target location. In some examples, the dilator tip 54 may taper in a distal direction and may be configured to taper from an axial location at the distal end 48 of the tube 26 of the guide extension catheter 14 to a distal end of the dilator tip 54 (e.g., the distal end of the dilator device 52) when the stopper 58 is engaged with the tube 26 and/or the collar 42. The tube 60 of the dilator device 52 may couple with a proximal end of the dilator tip 54 and a lumen of the tube 60 may be axially aligned with a lumen of the dilator tip 54. Alternatively or additionally, the tube 60 may extend into dilator tip 54 a desired distance (e.g., extend into the dilator tip 54 a partial length of the dilator tip 54 and then end proximal of the distal end of the dilator tip 54, extend to the dilator tip 54, through an entire length of the dilator tip 54, and terminate at a distal end of the dilator tip 54, etc.)

The dilator tip 54 may have a taper portion 54a and, optionally, an extension portion 54b extending in a proximal direction from the taper portion 54a. In some examples, the extension portion 54b may be omitted. The tube 60 may be configured to couple with the dilator tip at the taper portion 54a, the extension portion 54b, or both of the taper portion 54a and the extension portion 54b.

The taper portion 54a of the dilator tip 54 may have any suitable outer diameter. For example, the outer diameter of the taper portion 54a of the dilator tip 54 may be in a range of or vary between about 0.559 mm (0.022 inches) to about 1.702 mm (0.067 inches), but other suitable outer diameters are contemplated. In some examples, a maximum outer diameter of the taper portion 54a may be about 1.702 mm (0.067 inches), 1.448 mm (0.057 inches), 1.397 mm (0.055 inches), 1.245 mm (0.049 inches), and/or suitable values. In one example, a minimum outer diameter of the taper portion 54a may be about 0.432 mm (0.017 inches) and/or suitable values. In some examples, the taper portion 54a of the dilator tip 54 may have an outer diameter at a proximal end of the taper that is equal to or approximately equal to an inner diameter of the tube 26 of the guide extension catheter 14. In some examples, the dilator tip 54 may be flexible or naturally expandable when unconstrained such that the outer diameter at the proximal end of the taper portion 54a may be equal to or approximately equal to an outer diameter of the tube 26 of the guide extension catheter 14. The taper portion 54a of the dilator tip 54 may taper to a distal end of the dilator device 52 and an outer diameter at the distal end of the dilator tip 54 may be equal to or approximately equal to an inner diameter of a lumen extending through the dilator tip 54 and configured to receive the guidewire over which the dilator device 52 is configured to track. The taper from the proximal end of the taper portion 54a to the distal end of the taper portion 54a may be consistent or constant or a rate of change of taper may change at one or more locations. In some examples, a distal end of the guide extension catheter 14 may have a taper that tapers at a same or similar rate of change as a taper of the taper portion 54a at an axial location configured to be located proximate the distal end of the guide extension catheter 14. Such outer diameter configurations of the dilator tip 54a may facilitate the guide extension catheter system 50 traversing vessels while mitigating the likelihood of catching the distal end of the tube 26 on the vessel.

The taper portion 54a of the dilator tip 54 may have any suitable length. In some examples, a suitable length of the taper portion 54a may be a length sufficient to create a structural taper between an inner or outer diameter of the tube 26 of the guide extension catheter 14 and a guidewire to prevent or mitigate the likelihood of the guide extension catheter 14 catching on a vessel wall as the guide extension catheter 14 is advanced to a target location. Example suitable lengths of the taper portion 54a of the dilator tip 54 include, but are not limited to, lengths in a range of about 5 mm (0.1.97 inches) to about 45 mm (1.772 inches). In one example, the taper portion 54a may have a length of about 15 mm (0.591 inches). Other suitable lengths of the taper portion 54a are contemplated.

The extension portion 54b of the dilator tip 54 may have any suitable outer diameter. In one example, the outer diameter of the extension portion 54b of the dilator tip 54 may be in a range of 1.194 mm (0.047 inches) to about 1.397 mm (0.055 inches), but other suitable outer diameters are contemplated. In some examples, the extension portion 54b of the dilator tip 54 may have an outer diameter equal to or slightly less than the inner diameter of the tube 26 of the guide extension catheter 14 such that the extension portion 54b may longitudinally and/or rotationally translate within the tube 26. In some examples, the extension portion 54b may have a constant outer diameter along its length and in other examples, the outer diameter of the extension portion 54b may vary along the length of the extension portion 54b. In one example, an outer diameter of a proximal end of the extension portion 54b may be beveled or tapered in a proximal direction. In one example, an outer diameter of the extension portion 54b may form a longitudinally extending curve, longitudinally extending steps, and/or other suitable shapes or configurations.

When included, the extension portion 54b of the dilator tip 54 may have any suitable length. In some examples, the extension portion 54b may have a length configured to facilitate extending the dilator tip 54 (e.g., the taper portion 54a and the extension portion 54b of the dilator tip 54) a known or predetermined distance beyond a distal end of the guide extension catheter 14 when the stopper 58 is engaged with the tube 26 or collar 42 of the guide extension catheter 14. Example suitable lengths of the extension portion 54b of the dilator tip 54 include, but are not limited to, lengths in a range of about 20 mm (0.787 inches) to about 100 mm (3.937 inches). Other suitable lengths of the extension portion 54b are contemplated.

The dilator tip 54 may be formed from one or more suitable biocompatible materials. In some examples, the dilator tip 54 may be formed from a flexible material configured to flex when engaging a vessel wall, while maintaining sufficient column strength to traverse a vessel along a guidewire. The taper portion 54a and the extension portion 54b, when included, may be formed from the same material(s) or from different materials. Example suitable materials for forming the dilator tip 54 include, but are not limited to, the biocompatible materials discussed herein, metals, polymers, a rigid material, silicone, TPEs, a block copolymer, PEBAs (e.g., PEBAX®), polyethylenes, high density polyethylene (HDPE), and/or other suitable materials. In some examples, the dilator tip 54 may be formed from a single material in a single layer of materials, a single material in a plurality of layers of materials, or a plurality of materials in a plurality of layers. In one example, the dilator tip 54 may be formed from a single layer of (e.g., formed entirely from) PEBA. In one example, the dilator tip 54 may be formed with an inner layer of HDPE and an outer layer of PEBA. In one example, the dilator tip 54 may be entirely or at least partially formed from a PEBA material having a 45 Shore D durometer. It is contemplated that the dilator tip 54 may be formed from other suitable materials and/or with other suitable durometers.

The components of the dilator device 52 may be engaged and/or coupled together in any suitable manner. For example, the components of the dilator device 52 may be coupled together using over-molding techniques, extrusion techniques, thermal bonding techniques, reflow techniques, adhesive bonding techniques, friction fit techniques, and/or other suitable techniques. In some examples, the stopper 58 may be coupled to the proximal member 56 and the tube 60 with thermal bonding and/or reflow techniques and the dilator tip 54 may be coupled to the tube 60 with thermal bonding and/or reflow techniques, but other suitable coupling techniques are contemplated.

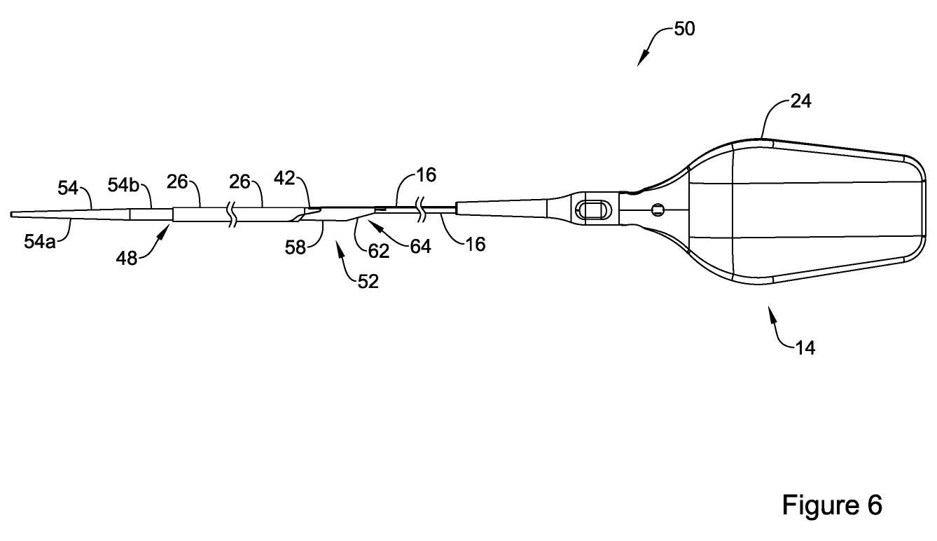

FIG. 6 depicts a schematic side view of the illustrative guide extension catheter system 50 depicted in FIG. 5, with the dilator device 52 coupled with the guide extension catheter 14. As depicted in FIG. 6, the dilator tip 54 has been inserted through the tube 26 of the guide extension catheter 14 with the stopper 58 extending into and engaging (e.g., with a friction fit) the tube 26 and/or collar 42 of the guide extension catheter 14. With the stopper 58 engaging the tube 26 and/or the collar 42, the dilator tip 54 may extend a known or predetermined distance distal of or beyond the distal end 48 of the guide extension catheter 14. Although other configurations are contemplated, the taper portion 54a and the extension portion 54b may extend beyond the distal end 48 of the guide extension catheter 14 with the stopper 58 engaging the tube 26 and/or the collar 42.

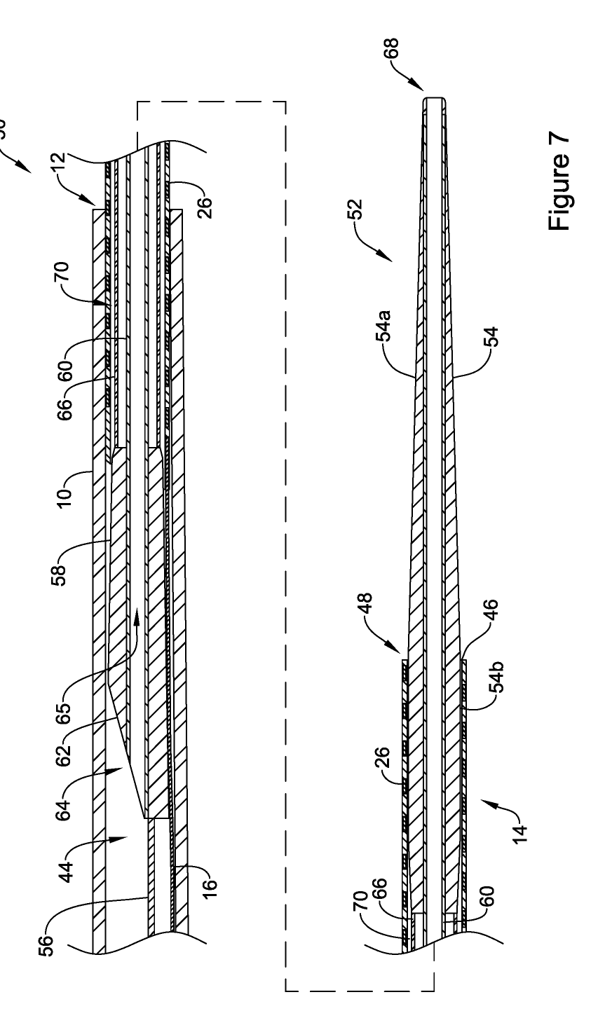

FIG. 7 depicts a schematic cross-section view of an illustrative configuration of the guide extension catheter system 50 extending through the guide catheter 10. As depicted in FIG. 7, the guide extension catheter system 50 may extend along the guide catheter 10 and distal of the distal end 12 of the guide catheter 10. In some examples, the proximal member 16 may extend proximal of the tube 26 and/or the collar 42 and along the guide catheter 10 to a location proximal of a proximal end of the guide catheter 10. In a similar manner, the proximal member 56 may extend proximal of the stopper 58 and along the guide catheter 10 to a location proximal of a proximal end of the guide catheter 10. With the stopper 58 engaging the tube 26 of the guide extension catheter 14, the dilator tip 54 may extend through the tube 26 and distal of the distal end 48 of the guide extension catheter 14. In some examples, a proximal end of the dilator tip 54 (e.g., an axial location having a greatest outer diameter) may be configured to be located at the distal end 48 of the guide extension catheter 14 when the stopper is engaged with the tube 26 and/or the collar 42.

As discussed, the stopper 58 may have any suitable outer surface configuration. In one example and as depicted in FIG. 7, the outer surface of the stopper 58 may define a distal taper configured to engage the tube 26 of the guide extension catheter 14 with a friction fit type of coupling. Other suitable configurations are contemplated.

As depicted in FIG. 7, the tube 60 of the dilator device 52 may at least partially define and extend from the guidewire port 64, define a guidewire lumen 65, and extend through the stopper 58 and through the dilator tip 54 to a location proximate a distal end 68 of the dilator device 52. Although the tube 60 is depicted as terminating proximal of the distal end 68 of the dilator device 52, in some examples the tube 60 may terminate at the distal end 68. The guidewire lumen 65 may extend through the stopper 58 and the dilator tip 54 and may or may not be defined by the tube 60.

The dilator device 52 may include one or more outer layers or coatings 66 as part of and/or extending along the tube 60. As depicted in FIG. 7, the outer layer or coating 66 may extend from a distal end of the stopper 58 to a proximal end of the dilator tip 54. The outer layer or coating 66 may extend between one or more other axial locations while extending over the tube 60. The outer layer or coating 66 may be a coating or layer applied to an outer surface of the tube 60 and/or a tube layer extending over the tube 60. Although the outer layer or coating 66 is depicted in FIG. 7 as being spaced from an outer surface of the tube 60, the outer layer or coating 66 may be applied directly to or may contact the outer surface of the tube 60.

When the stopper 58 of the dilator device 52 is engaging the tube 26 or collar 42 and the dilator tip 54 is extending out of the distal end 48 of the guide extension catheter 14, a fluid or air pocket 70 may be created within the guide extension catheter system 50. For example, the fluid or air pocket 70 may be defined by an outer surface of the tube 60 or the outer layer or coating 66, an inner surface of the tube 26 of the guide extension catheter, the stopper 58, and the dilator tip 54. Other locations of the fluid or air pocket 70 are contemplated.

The guide extension catheter 14 and the dilator device 52 may be inserted into and/or through the guide catheter 10 simultaneously and/or otherwise with one another. When inserted together through the guide catheter 10, the dilator device 52 may be inserted into and through the guide extension catheter 14 such that the stopper 58 is engaging the tube 26 and the dilator tip 54 is extending through tube 26 prior to inserting the guide extension catheter system 50 through the guide catheter 10 and into a patient. As such, air and/or other suitable fluids (e.g., gasses, liquids, etc.) may be trapped within the fluid or air pocket 70 and when the dilator device 52 is withdrawn from the guide extension catheter 14 (e.g., after the guide extension catheter 14 is positioned at or proximate a target location) to insert a treatment device through the guide extension catheter 14, air or other fluid in the fluid or air pocket 70 may be inadvertently released into the vessel of the patient and the air or other fluid may cause a clot or embolism or other adverse condition in the patient.

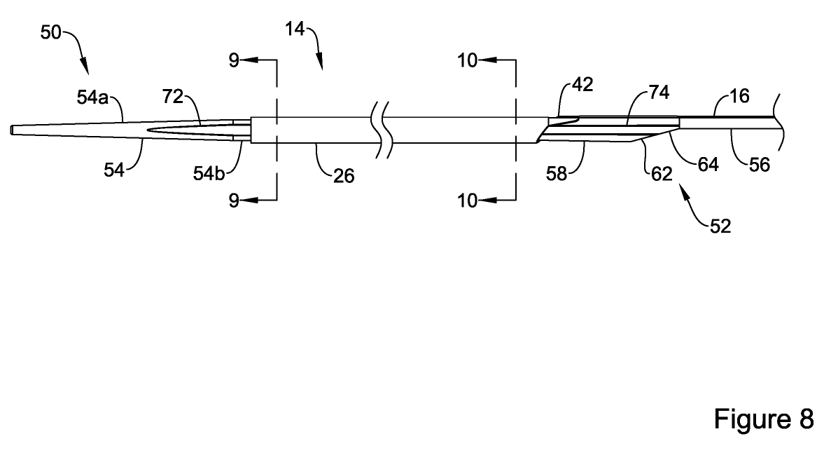

To prevent or mitigate a likelihood of a fluid or air pocket from forming within the guide extension catheter system 50, one or both of the stopper 58 and the dilator tip 54 may include a circumferential profile configured to allow air or fluid to escape from a location within the tube 26 of the guide extension catheter 14 between the stopper 58 and the dilator tip 54. FIG. 8, for example, depicts a schematic side view of an illustrative configuration of the guide extension catheter system 50 configured to allow fluid (e.g., blood, air, etc.) to flow along the dilator tip 54 and/or the distal stopper 58 when the distal stopper 58 is engaged with the tube 26 and/or the collar 42 of the guide extension catheter 14.

One or both of the dilator tip 54 and the stopper 58 may have profiles (e.g., circumferential profiles) configured to purge fluid between the dilator tip 54 and the distal stopper 58 and allow blood to pass therethrough when the distal stopper 58 is engaged with the tube 26 of the guide extension catheter 14. As depicted in FIG. 8, the dilator tip 54 may include one or more recesses 72 (e.g., a recess extending radially inward) and/or the stopper 58 may include one or more recesses 74 (e.g., a recess extending radially inward) that at least partially define a profile of an outer surface of the dilator tip 54 and/or the stopper 58. Although one or both of the dilator tip 54 and the stopper 58 may include only a single recess 72, 74, one or both of the dilator tip 54 and the stopper 58 include a plurality of recesses 72, 74. In some examples, the one or more recesses 72 may extend from a proximal end of the dilator tip 54 to a location distal of the distal end 48 of the guide extension catheter 14 when the stopper 58 is engaging the tube 26 so as to place the space between the dilator tip 54 and the stopper 58 in fluid communication with the exterior of the guide extension catheter system 50 (e.g., with a space exterior of the guide extension catheter system 50). In some examples, the one or more recesses 74 may extend from a distal end of the stopper 58 to a location proximal of a proximal end of the tube 26 or other suitable location when the stopper 58 is engaging the tube 26 so as to place the space between the dilator tip 54 and the stopper 58 in fluid communication with the exterior of the guide extension catheter system 50.

The recesses 72, 74 may be any suitable type recesses configured to place the space between the dilator tip 54 and the stopper 58 in fluid communication with the exterior around the guide extension catheter system 50 when the stopper 58 is engaging the tube 26. For example, the recesses 72, 74 may be indents, spaces between protrusions, tubes, lumens, and/or other suitable types of recesses. The recesses may have any suitable shapes including, but not limited to, one or more lines spaced from one another, circles, rectangles, oblong shapes, and/or other suitable shapes. In one example configuration of the dilator device 52, the dilator tip 54 may have a first profile with a recess 72 at a first circumferential location and a recess 72 at a second circumferential location, where the second circumferential location is diametrically opposed to the first circumferential location and/or the stopper 58 may have a second profile with a recess 74 at a first circumferential location and a recess 74 at a second circumferential location, where the second circumferential location is diametrically opposed to the first circumferential location. In the example, when the stopper 58 is engaged with tube 26 or collar 42, the first profile may allow fluid to enter and/or exit through a distal end of the tube 26 and the second profile may allow fluid to enter or exit through a proximal end of the tube 26.

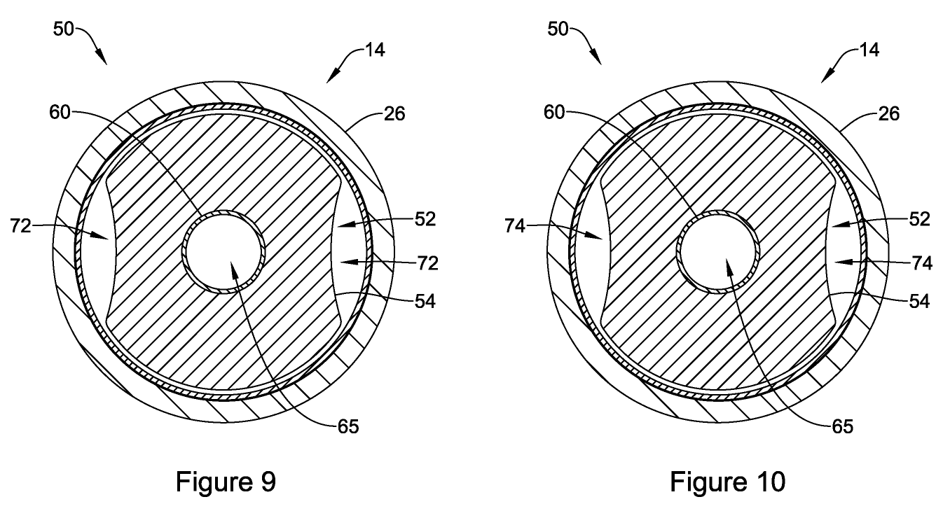

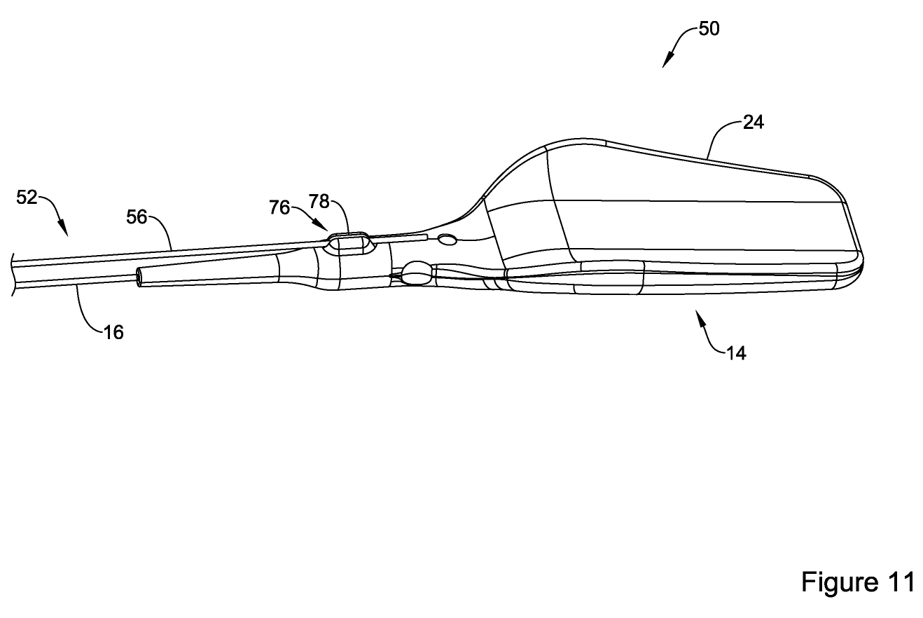

FIGS. 9 and 10 depict schematic cross-section views of the guide extension catheter system 50 with the dilator tip 54 having the first profile and stopper 58 having the second profile, take along line 9-9 and line 10-10, respectively, in FIG. 8. As depicted in FIG. 9, the first profile with the recesses 72 may create space between an outer surface of the dilator tip 54 and an inner surface of the tube 26 to allow fluid to pass along the dilator tip 54 when the stopper 58 is engaged with the tube 26. Similarly, and as depicted in FIG. 10, the second profile with the recesses 74 may create space between an outer surface of the stopper 58 and an inner surface of the tube 26 to allow fluid to pass along the dilator tip 54 when the stopper 58 is engaged with the tube 26. Although the first profile of the dilator tip 54 depicted in FIG. 9 includes two of the recesses 72 formed as indentations and diametrically opposed and the second profile of the stopper 58 depicted in FIG. 10 two of the recesses 74 formed as indentations and diametrically opposed, other suitable number of the recesses 72 and/or the recesses 74 may be used. Additional and/or other suitable configurations of the first profile and/or the second profile are contemplated.

To prevent the guide extension catheter 14 and the dilator device 52 of the guide extension catheter system 50 from entangling one another and/or to facilitate delivering the guide extension catheter system 50 to a target location in a vessel of a patient, the guide extension catheter system 50 may include one one or more connectors configured to maintain a connection between the guide extension catheter 14 and the dilator device 52 during advancement of the guide extension catheter system 50 to a target location, configured to prevent or mitigate tangling of the guide extension catheter 14 and the dilator device 52 during advancement of the guide extension catheter system 50 through a vessel, configured to prevent or mitigate unintended relative motion of the guide extension catheter 14 and the dilator device 52 during advancement of the guide extension catheter system 50 through a vessel, configured to maintain alignment of the guide extension catheter 14 and the dilator device 52 during advancement of the guide extension catheter system 50 through a vessel, and/or configured to provide other suitable benefits during storage and/or use of the guide extension catheter system 50. Example suitable connectors for coupling the proximal portion of the guide extension catheter 14 with the proximal portion of the dilator device 52 include, but are not limited to, clips, sleeves, clasps, holders, friction fit seals, locks, and/or other suitable connectors.

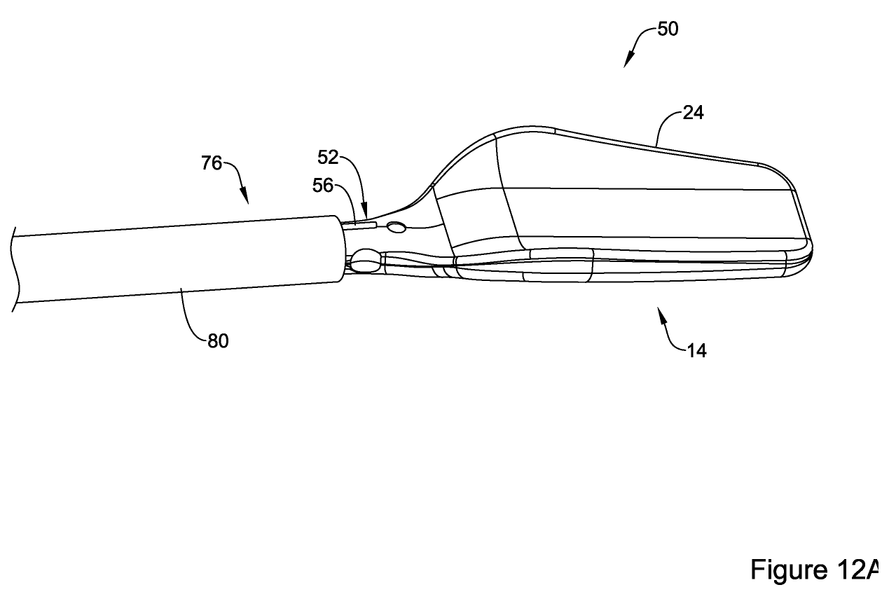

FIG. 11 depicts a schematic perspective of a proximal portion of the guide extension catheter system 50. As depicted in FIG. 11, the hub 24 of the guide extension catheter 14 may be coupled with the proximal member 16 with a connector 76. The connector 76 may be configured to axially and/or rotationally fix the dilator device 52 relative to the guide extension catheter 14. The connector 76 may be a clip 78 configured to receive and engage the proximal member 56 of the dilator device 52. In some examples, the clip 78 may include one or more arms or fingers configured to receive the proximal member 56 therebetween and provide a compression force on the received proximal member 56 and prevent or mitigate unintended axial and/or rotational translation of the proximal member 56. In some examples, the clip 78 may be formed as part of the hub 24, as depicted for example in FIG. 11, but the clip 78 or other connector 76 may be formed as part of the dilator device 52 and/or as a component separate from the guide extension catheter 14 and the dilator device 52. Other suitable configurations of the connector 76 and/or the clip 78 are contemplated.

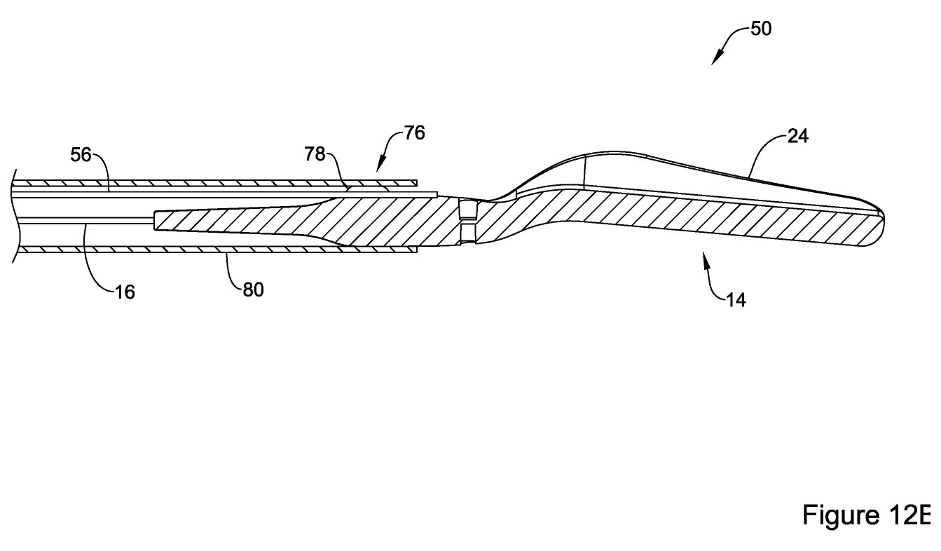

FIGS. 12A and 12B depict schematic views of the proximal portions of the dilator device 52 and the guide extension catheter 14 coupled together similar to as depicted in FIG. 11, but with a sleeve 80 extending over the clip 78 and facilitating the coupling between the dilator device 52 and the guide extension catheter 14 to a maintain a relative position therebetween. FIG. 12A depicts a schematic perspective view of the sleeve 80 extending over the proximal member 56 of the dilator device 52, the proximal member 16 (not shown in FIG. 12A) of the guide extension catheter 14, the clip 78 (not shown in FIG. 12A), and the hub 24. FIG. 12B depicts a schematic cross section view of the sleeve 80 extending over the proximal member 56, the proximal member 16, the clip 78, and the hub 24. As depicted in FIG. 12B, the sleeve 80 may be configured to engage the clip 78 and a portion of the hub 24 of the guide extension catheter 14 to create friction fit coupling between the hub 24 and the sleeve 80 to facilitate maintaining the proximal member 56 engaged with the clip 78. Other suitable placements of the sleeve 80 relative to the guide extension catheter 14 and the dilator device 52 that facilitate coupling the guide extension catheter 14 with the dilator device 52 are contemplated.

The sleeve 80 may be part of the connector 76 or separate from the connector 76 and the sleeve 80 may be configured to facilitate maintaining the connection and/or a desired position between the dilator device 52 and the guide extension catheter 14. In some examples, the sleeve 80 may be a carrier tube, but other suitable types of sleeves are contemplated including, but not limited to, compression sleeves, an elongated clip, a packaging sleeve, a heat-sealed sleeve, and/or other suitable types of sleeves.

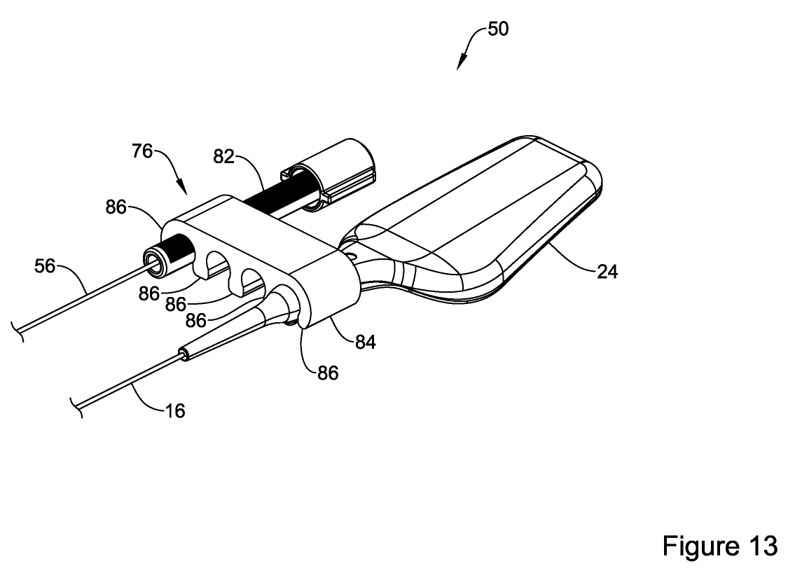

FIG. 13 depicts a schematic perspective view of a proximal portion of the guide extension catheter system 50. As depicted in FIG. 13, the dilator device 52 may include a hub 82 defining a proximal end of the dilator device 52 and coupled with the proximal member 56. An illustrative connector 76 may be configured to receive or engage the hub 82 of the dilator device 52 and the hub 24 (e.g., a neck of the hub 24) of the guide extension catheter 14 to couple the dilator device 52 with the guide extension catheter 14.

The connector 76 depicted in FIG. 13 may be configured as a clasp 84. The clasp 84 may be separate from and configured to be coupled with both of the dilator device 52 and the guide extension catheter 14. Alternatively, the clasp 84 may be permanently coupled with one of the dilator device 52 and the guide extension catheter 14 and configured to connect or couple with the other. The clasp 84 may include two or more arms 86 configured to apply a compression force to a component received therein. The hub 82 of the dilator device 52 and the hub 24 of the guide extension catheter 14 may each be received between two adjacent arms 86. In some examples, the arms 86 may be configured to clip onto the hub 82, the hub 24, and/or other suitable components of or usable with (e.g., ancillary devices, treatment devices, etc.) the guide extension catheter system 50.

The arms 86 of the clasp 84 may include one or more flexible or resilient arms 86 configured to flex or adjust to change a space therebetween for receiving and engaging (e.g., holding or grasping) a component of or usable with the guide extension catheter system 50. In some examples, all of the arms 86 of the clasp 84 may be flexible or resilient to receive and engage components of or be usable with the guide extension catheter system 50. Alternatively, one or more (e.g., one, two, all) of the arms 86 may be inflexible or rigid. In some examples, when at least one of the arms 86 is inflexible, the inflexible arms 86 may be adjacent to a flexible arm 86 such that the spacing between arms 86 may be adjusted to receive and engage the component inserted between adjacent arms 86, but other suitable configurations are contemplated.

The clasp 84 may have any suitable number of arms 86 configured to receive any suitable number of components or portions of the guide extension catheter system 50 and/or components useable with the guide extension catheter system 50. For example, the clasp 84 may be configured to receive or engage at least two components and may include three arms defining two openings to receive components, four arms defining three openings to receive components, five arms defining four openings to receive components, six arms defining five openings to receive components, and/or other suitable number of arms defining openings to receive components.

The arms 86 may be spaced from one another any suitable distance configured to receive a component of or used with the guide extension catheter system 50. In some examples, the arms 86 of the clasp 84 may be spaced an equidistance away from one another. Alternatively, in some examples one or more of the arms 86 may be spaced a different distance away form an arm 86 than one or more other arms 86 are space away from an arm 86. In some examples, a space between two arms 86 may be configured to engage a component having a particular diameter. In one example, a space between two arms 86 configured to receive the hub 82 of the dilator device 52 or the hub 24 of the guide extension catheter 14 may span a distance different than a distance between two arms 86 configured to receive the proximal member 56 of the dilator device 52 or the proximal member 16 of the guide extension catheter 14. Other suitable configurations of the spacing between arms 86 of the clasp 84 are contemplated.

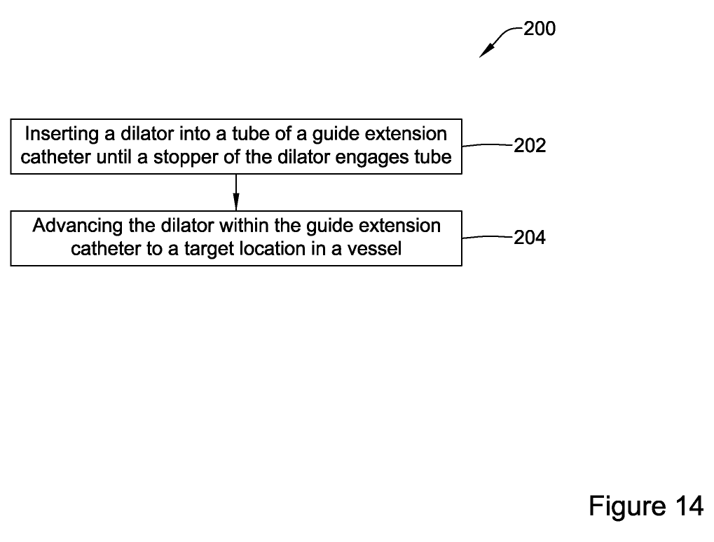

FIG. 14 depicts a schematic diagram of a method 200 of using a guide extension catheter system. The method 200 may utilize the guide extension catheter system 50 described herein or one or more other suitable guide extension catheter systems. In some examples, the guide extension catheter system may consist of or include at least a guide extension catheter and a dilator device.

The method 200 may include inserting 202 a dilator device into a tube or distal sheath of a guide extension catheter until a stopper of the dilator device engages the distal sheath or a collar of the guide extension catheter. The dilator device may include a distal tip coupled with and spaced in a distal direction from the stopper. In some examples, the distal tip may be flexible, tapered in a distal direction, and configured to track over a guidewire through tortuous vessels. When the stopper is engaged with the distal sheath or collar of the guide extension catheter, the distal tip with the taper may extend in a distal direction a desired distance (e.g., a predetermined or known distance) from the distal end of the guide extension catheter (e.g., from the distal end of the distal sheath).

The method 200 may include advancing 204 the guide extension catheter and the dilator device to a target location in a blood vessel of a patient while the distal stopper is engaged with the distal tube. With the distal tip being flexible and having a distal taper, the guide extension catheter engaged with the dilator device may be tracked over a guidewire extending through one or both of the dilator tip and the stopper (e.g., through a guidewire lumen defined by the dilator device and a guidewire port defined by or at a proximal end of the distal stopper) to the target location, while preventing or mitigating a distal end of the guide extension catheter from catching on a vessel wall or an ostium of side branch vessels extending from a vessel in which the guide extension catheter system is traversing. Although other configurations are contemplated, the guide extension catheter system 50 may be advanced along the guidewire and through a guide catheter, where the guidewire may extend distally of a distal end of the guide catheter.

Once the guide extension catheter system has reached the target location in a vessel, the dilator device may be withdrawn from the guide extension catheter and the guide extension catheter may be maintained at or proximate the target location. In some examples, the dilator device may be entirely withdrawn from the patient. After withdrawing the dilator device, a treatment device may be inserted into the patient, advanced over the guidewire, and through the guide extension catheter (e.g., through the distal sheath of the guide extension catheter) to the target location in the vessel and a treatment may be performed with the treatment device at the target location.

While the stopper of the dilator device is engaged with the distal sheath and/or the collar of the guide extension catheter, fluid may be allowed to flow between an interior of the distal sheath between a space extending from the dilator tip to the stopper and an exterior of the distal sheath. Allowing fluid to flow from within the sheath to the exterior of the sheath while the stopper is engaged with the distal sheath or collar may prevent air or other fluid form being trapped between the distal tip and the stopper so as to prevent or mitigate causing an embolism in the patient due to an inadvertent release of air or fluid within the vessel when the dilator device is withdrawn or removed from the guide extension catheter and the patient. In some example configurations, one or both of the dilator tip and the distal stopper may include one or more recesses configured to allow fluid to flow between the interior of the distal sheath between the dilator tip and the stopper and the exterior of the distal sheath while the stopper is engaged with the distal tube, as discussed herein or in other suitable configurations.

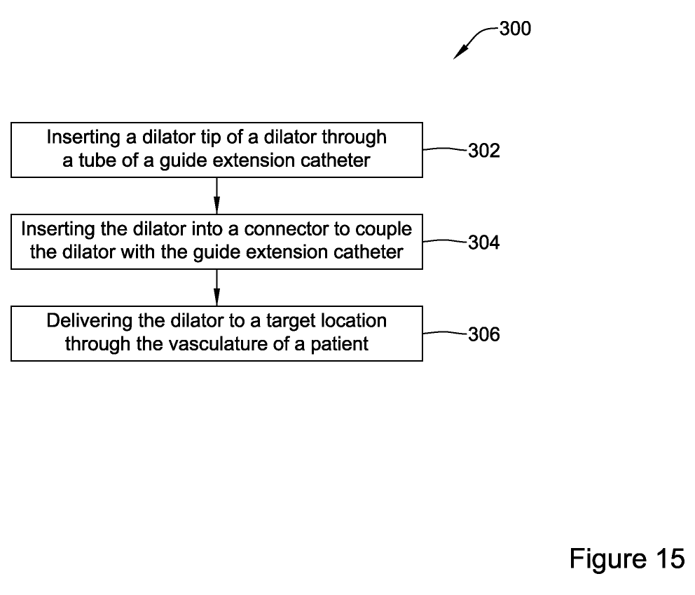

FIG. 15 depicts a schematic diagram of a method 300 of using a guide extension catheter system. In some examples, the method 300 or portions of the method 300 may be part of or be usable with the method of 200. The method 300 may utilize the guide extension catheter system 50 described herein or one or more other suitable guide extension catheter systems. In some examples, the guide extension catheter system may consist of or include at least a guide extension catheter and a dilator device. In some examples, the guide extension catheter system may include or be used with a connector

The method 300 may include inserting 302 a dilator device into a tube or distal sheath of a guide extension catheter. In some examples, the dilator device may be inserted into the distal sheath until a stopper of the dilator device engages the distal sheath or a collar of the guide extension catheter. The dilator device may include a distal tip coupled with and spaced in a distal direction from the stopper and a proximal member (e.g., a dilator pusher) coupled with and extending in a proximal direction from the stopper. In some examples, the dilator device may include a handle or hub at a proximal end of the proximal member. The guide extension catheter may include a shaft extending in a proximal direction from the distal sheath. In some examples, the guide extension catheter may include a hub or a handle (e.g., a guide extension hub) at the proximal end of the shaft.

The method 300 may include inserting 304 the dilator device into a connector to couple the dilator device with the guide extension catheter. The connector may be any suitable type of connector configured to facilitate coupling the dilator device with the guide extension catheter including, but not limited to, the types of connectors described herein and/or other suitable connectors. In some examples, the connector may be part of the hub or handle of the guide extension catheter and inserting 304 the dilator device into the connector may include inserting the proximal member of the dilator device into the connector. In some examples, inserting 304 the dilator into the connector may include connecting the handle or hub of the dilator device with the connector (e.g., inserting the hub into the connector) and connecting the hub or handle of the guide extension catheter with the connector (e.g., inserting the handle into the connector) to couple the dilator device with the guide extension catheter. Other suitable types of connectors and connector insertion techniques are contemplated.

The method 300 may include delivering 306 the dilator device to a target location in a blood vessel of a patient while the dilator device is coupled with the guide extension catheter using the connector. In some examples, the guide extension catheter may be delivered to the target location with dilator device. The connector may facilitate maintaining a relative or desired position between the dilator device and the guide extension catheter as the dilator device and the guide extension catheter are delivered to the target location. In some examples, delivering 306 the dilator device to the target location may include tracking the dilator device over a guidewire extending through one or both of the dilator tip and the stopper (e.g., through a guidewire lumen defined by the dilator device and a guidewire port defined by or at a proximal end of the distal stopper) to the target location. Although other configurations are contemplated, the guide extension catheter system may be advanced along the guidewire and through a guide catheter, where the guidewire may extend distally of a distal end of the guide catheter.

Once the guide extension catheter system (e.g., at least the dilator device) has reached the target location in a vessel, the dilator device may be withdrawn from the guide extension catheter and the guide extension catheter may be maintained at or proximate the target location. In some examples, the dilator device may be entirely withdrawn from the patient. After withdrawing the dilator device, a treatment device may be inserted into the patient, advanced over the guidewire, and through the guide extension catheter (e.g., through the distal sheath of the guide extension catheter) to the target location in the vessel and a treatment may be performed with the treatment device at the target location.

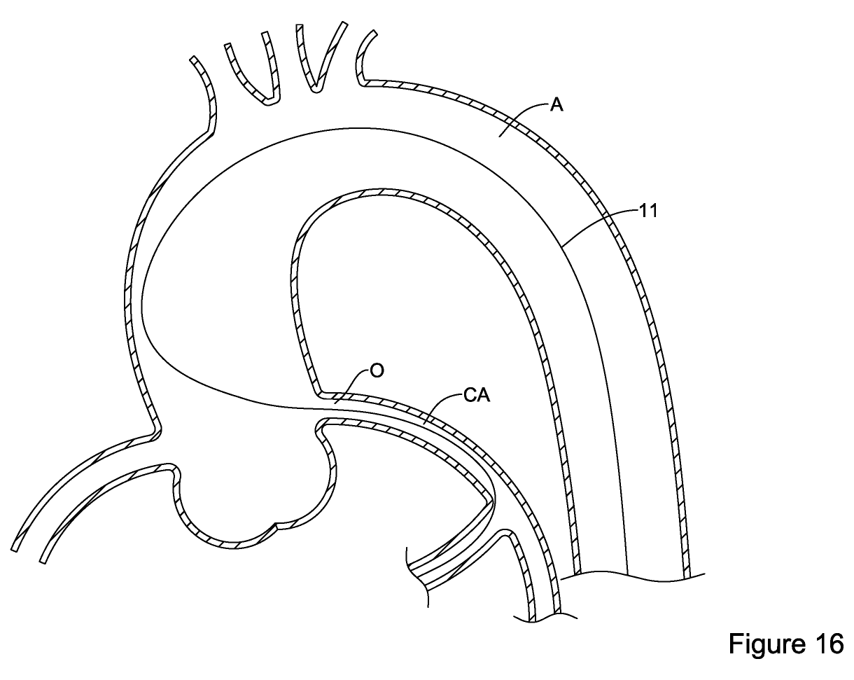

FIGS. 16-23 depict schematic diagrams of steps in using the guide extension catheter system 50 described herein to reach a target location and perform a treatment at the target location. In one example and as depicted in FIGS. 16-23, the treatment may be a minimally-invasive cardiac intervention such as a percutaneous transluminal coronary angioplasty. Although FIGS. 16-23 depict and are described with respect to components of the guide extension catheter system 50, other suitable components may be utilized to reach the target location and/or treat the patient at the target location.

FIG. 16 depicts a schematic view of a guidewire 11 advanced through a blood vessel, such as the aorta A, to at least a target location. The guidewire 11 may be percutaneously inserted into the vasculature of the patent and advanced to the target location using standard guidewire positioning techniques.

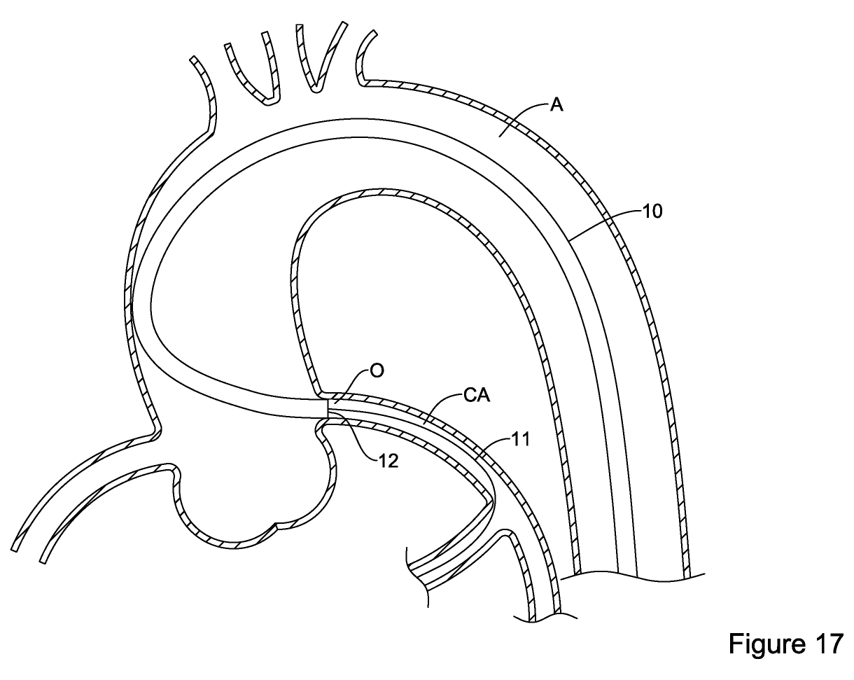

FIG. 17 depicts a schematic view of the guide catheter 10 advanced through the aorta A to a position adjacent to the ostium O of a (e.g., left and/or right) coronary artery CA. In some examples, the guide catheter 10 may be advanced to the ostium O over the guidewire 11.

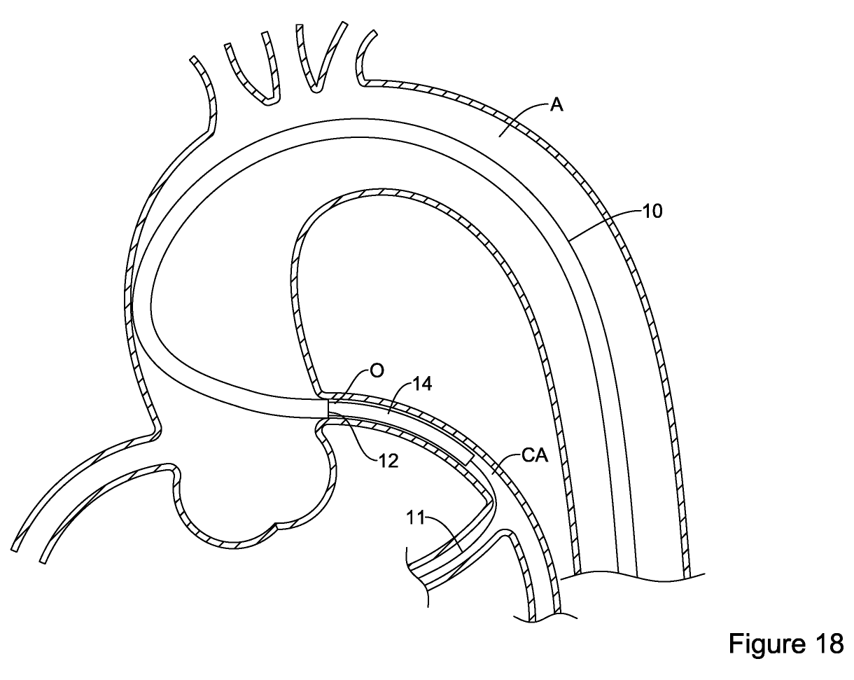

FIG. 18 depicts a schematic view of the guide extension catheter 14 extending through the guide catheter 10 and over the guidewire 11 beyond the distal end 12 of the guide catheter 10 into the coronary artery CA. Because, for example, the guide extension catheter 14 may extend beyond the distal end 12 of the guide catheter 10, the guide extension catheter 14 may extend beyond the ostium O of the coronary artery CA and into a portion of the coronary artery CA. By extending beyond the ostium O, the guide extension catheter 14 may stabilize the positioning of the guide catheter 10 and allow for improved access to the coronary artery CA for a number of cardiac interventions. However, due to narrowing of a margin between an outer diameter of the guide extension catheter 14 and the vessel as the guide extension catheter 14 is advanced distally, a difference in diameter between the guidewire 11 and the outer diameter of the guide extension catheter 14, and a need to enter vessels branching off of the coronary artery CA, there is a risk that the distal end of the guide extension catheter 14 may catch on a wall of the coronary artery CA or an ostium leading to a side branch vessel if the guide extension catheter 14 were to track over the guidewire 11 to the target location.

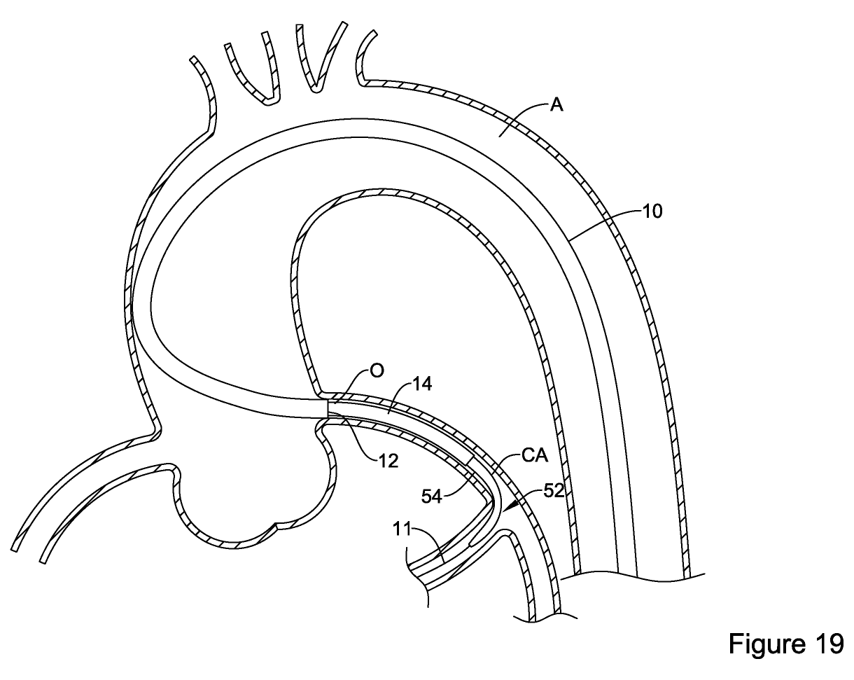

FIG. 19 depicts the dilator device 52 advanced over the guidewire 11 to a target location in a side branch vessel extending from the coronary artery CA. The dilator tip 54 of the dilator device 52 may extend distally of the distal end of the guide extension catheter 14 and may be sufficiently flexible to track along the guidewire 11 around a corner between a first vessel (e.g., the coronary artery CA) and a second vessel extending from the first vessel. The dilator tip 54 may include the taper discussed herein creating a gradual increase in diameter from a diameter of the guidewire 11 to an inner or outer diameter of the guide extension catheter 14. Although FIGS. 18 and 19 depict the dilator device 52 being inserted through the guide extension catheter 14 after the guide extension catheter 14 has been inserted into the coronary artery CA, this is not required and the dilator device 52 may be inserted through and/or coupled with the guide extension catheter 14 prior to the guide extension catheter 14 being inserted into the patient such that the guide extension catheter 14 and the dilator device 52 may be advanced through the vasculature of the patient together.

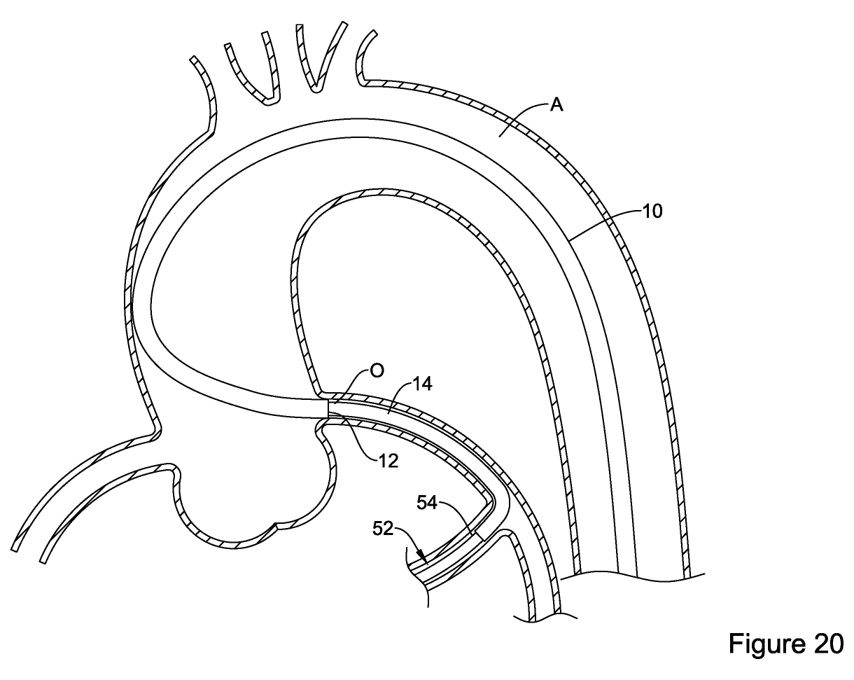

FIG. 20 depicts a schematic view of the dilator tip 54 of the dilator device 52 advanced over the guidewire 11 with the guide extension catheter 14 such that the distal end of the guide extension catheter 14 is at the target location and with a sheath or tube of the guide extension catheter 14 extending proximally to a location within the guide catheter 10. Because the dilator tip 54 has a proximal outer diameter equal to or substantially equal to the inner or outer diameter at the distal end of the guide extension catheter 14 and the outer diameter of the dilator tip 54 tapers distally, the risk of the distal end of the guide extension catheter 14 catching on the vessel walls is mitigated.

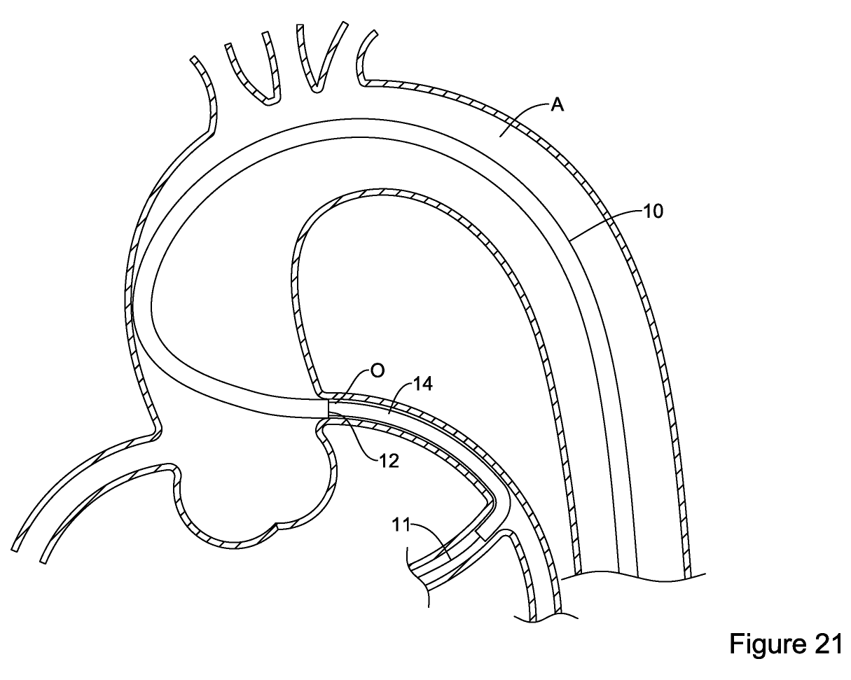

Once the guide extension catheter 14 is positioned at the treatment location, the dilator device 52 may be withdrawn from the patient, as depicted for example in the schematic view of FIG. 21. After the dilator device 52 has been removed from the patient, the guide extension catheter 14 may remain at or proximate the target location awaiting a treatment device or other suitable ancillary device (e.g., a balloon catheter, a stent delivery system, etc.)

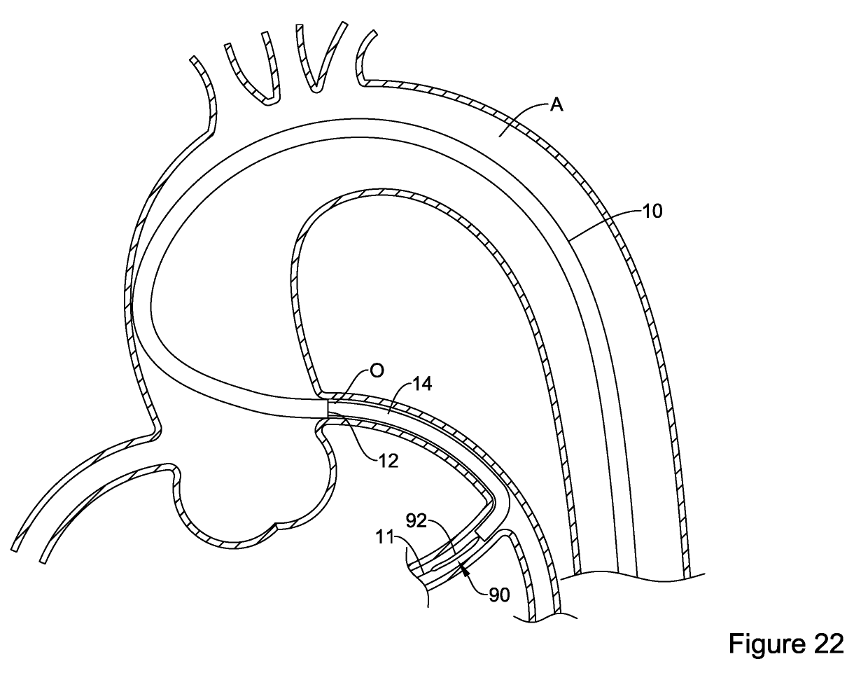

FIG. 22 depicts a schematic view of a treatment device 90 delivered to the target location through the guide catheter 10, through the guide extension catheter 14, and over the guidewire 11. In some examples, the treatment device 90 may be a balloon catheter with a balloon 92 and configured to perform a balloon angioplasty and/or deploy a stent.

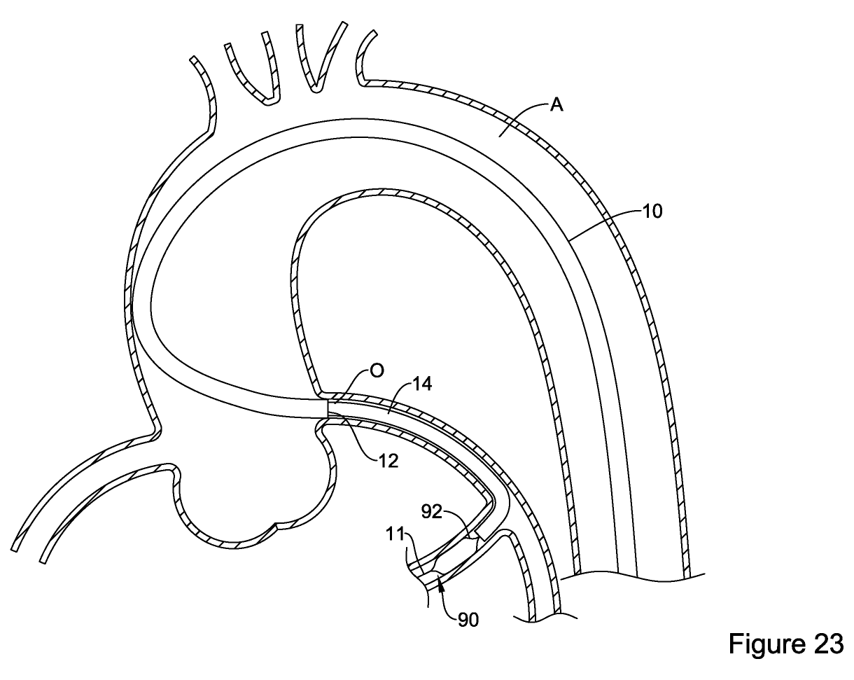

FIG. 23 depicts a schematic view of the treatment device 90 with the balloon 92 expanded (e.g., inflated) to perform a treatment within the vessel. Once the treatment has been performed, the balloon 92 may be deflated and withdrawn from the patient. Additional and/or alternative treatments may be performed on the patient using the guide extension catheter 14. After all treatments have been performed, the guide extension catheter 14, the guide catheter 10, and the guidewire 11 may be removed from the patient.

The materials that can be used for the various components of the guide extension catheters disclosed herein may vary. For simplicity purposes, the following discussion may reference components of the guide extension catheter system 50, however, this is not intended to limit the devices and methods described herein, as the discussion may be applied to other similar components or devices disclosed herein.

Components of the guide extension catheter system 50 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated "linear elastic" or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial "superelastic plateau" or "flag region" in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (which can also be distinguished based on its composition), which may accept only about 0.2 to 0.44 percent strain before plastically deforming.

In some configurations, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some configurations, there may be no martensite/austenite phase changes detectable by DSC and DMTA analysis in the range of about –60 degrees Celsius (ºC) to about 120 ºC in the linear elastic and/or non-super-elastic nickel-titanium alloy. The mechanical bending properties of such material may therefore be generally inert to the effect of temperature over this very broad range of temperature. In some configurations, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some configurations, the linear elastic and/or non-super-elastic nickel-titanium alloy may be in the range of about 50 to about 60 weight percent nickel, with the remainder being essentially titanium. In some configurations, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Patent Nos. 5,238,004 and 6,508,803, which are incorporated herein by reference. Other suitable materials may include ULTANIUM™ (available from Neo-Metrics) and GUM METAL™ (available from Toyota). In some other configurations, a superelastic alloy, for example a superelastic nitinol can be used to achieve desired properties.

In at least some configurations, portions or all of components of the guide extension catheter system 50 may be loaded with, made of, or otherwise include a radiopaque material. Radiopaque materials are understood to be materials capable of producing a relatively bright image on a fluoroscopy screen or another imaging technique during a medical procedure. This relatively bright image may aid the user of guide extension catheter system 50 in determining a location of components thereof. Some examples of radiopaque materials can include, but are not limited to, gold, platinum, palladium, tantalum, tungsten alloy, polymer material loaded with a radiopaque filler (e.g., barium sulfate, bismuth subcarbonate, etc.), and the like. Additionally, other radiopaque marker bands and/or coils may also be incorporated into the design of the components of the guide extension catheter system 50 to achieve the same result.

In some configurations, a degree of Magnetic Resonance Imaging (MRI) compatibility is imparted into the components of the guide extension catheter system 50. For example, the components of the guide extension catheter system 50, or portions thereof, may be made of a material that does not substantially distort the image and create substantial artifacts (i.e., gaps in the image). Certain ferromagnetic materials, for example, may not be suitable because they may create artifacts in an MRI image. The components of the guide extension catheter system 50, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In at least some configurations, a sheath or covering (not shown) may be disposed over portions of or all of one or more components of the guide extension catheter system 50 that may define a generally smooth outer surface. In other configurations, however, such a sheath or covering may be absent from a portion of all of the components of the guide extension catheter system 50. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some configurations the sheath can be blended with a liquid crystal polymer (LCP). For example, the mixture can contain up to about 6 percent LCP.

In some configurations, the exterior surface of one or more components of the guide extension catheter system 50 may be sandblasted, beadblasted, sodium bicarbonate-blasted, electropolished, etc. In these as well as in some other configurations, a coating, for example a lubricious, a hydrophilic, a protective, or other type of coating may be applied over portions of or all of the sheath or other surfaces of the components of the guide extension catheter system 50. Alternatively, the sheath may comprise a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Patent Nos. 6,139,510 and 5,772,609, which are incorporated herein by reference.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. These materials can vary widely without deviating from the scope of the present invention.

What is claimed is:

1. A guide extension catheter system comprising:
a guide extension catheter having a proximal end, a distal end, a guide extension hub at a proximal end, and a guide extension tube at the distal end; and
a dilator configured to be inserted within the guide extension tube, the dilator comprising:
a dilator pusher;
a dilator tip defining a distal end of the dilator; and
a distal stopper at a location proximal of the dilator tip and coupled with the dilator pusher,
wherein the guide extension hub is configured to couple with the dilator pusher.

2. The guide extension catheter system of claim 1, wherein the guide extension hub of the guide extension catheter comprises a connector configured to receive and engage the dilator pusher.

3. The guide extension catheter system of claim 2, wherein the connector is configured to axially fix the dilator relative to the guide extension catheter.

4. The guide extension catheter system of claim 1, wherein the distal stopper is configured to engage the guide extension tube.

5. The guide extension catheter system of claim 1, wherein the dilator tip is configured to extend from a distal end of the guide extension tube to a location distal of the distal end of the guide extension tube when the distal stopper is engaged with the guide extension tube and the guide extension hub is coupled with the dilator pusher.

6. The guide extension catheter system of claim 1, further comprising:
a carrier tube configured to extend over the guide extension hub and the dilator pusher.

7. The guide extension catheter system of claim 6, wherein the guide extension hub comprises a connector configured to engage the dilator pusher and the carrier tube is configured to extend over the connector when the connector is engaging the dilator pusher.

8. The guide extension catheter system of claim 6, wherein the guide extension catheter comprises a guide extension pusher extending between the guide extension hub and the guide extension tube, and the carrier tube is configured to extend over the guide extension pusher, the guide extension hub, and the dilator pusher.

9. The guide extension catheter system of claim 1, wherein the dilator comprises a dilator hub coupled with a proximal end of the dilator pusher.

10. The guide extension catheter system of claim 9, further comprising:
a connector,
wherein the guide extension hub is configured to couple with the dilator pusher via the connector.

11. The guide extension catheter system of claim 10, wherein the connector is configured to create a clip connection with the dilator hub and the guide extension hub.

12. A method of using a guide extension catheter system, the method comprising:
inserting a dilator tip of a dilator through a tube of a guide extension catheter, wherein the guide extension catheter comprises the tube and a guide extension hub and the dilator comprises the dilator tip and a dilator pusher;
inserting the dilator into a connector to couple the dilator with the guide extension catheter; and
delivering the dilator to a target location through vasculature of a patient while the dilator is coupled with the guide extension catheter using the connector.

13. The method of claim 12, further comprising:
engaging a dilator stopper with the tube of the guide extension catheter.

14. The method of claim 12, wherein the connector is part of the guide extension hub and inserting the dilator into the connector to couple the dilator with the guide extension catheter comprises inserting the dilator pusher into the connector.

15. The method of claim 12, wherein inserting the dilator into the connector to couple the dilator with the guide extension catheter comprises connecting a dilator hub coupled to the dilator pusher with the connector and connecting the guide extension hub with the connector.

16. The method of claim 12, further comprising:
removing the dilator tip from the tube of the guide extension catheter; and
delivering a treatment device through the tube of the guide extension catheter to the target location.

17. The method of claim 12, wherein delivering the dilator to the target location comprises delivering the dilator to the target location over a guidewire extending through the dilator tip and a dilator stopper of the dilator.

18. A guide extension catheter system comprising:
a guide extension catheter having a proximal end, a distal end, a guide extension hub at a proximal end, and a guide extension tube at the distal end; and
a dilator configured to be inserted within the guide extension tube, the dilator comprising:
a dilator pusher;
a dilator tip defining a distal end of the dilator; and
a distal stopper at a location proximal of the dilator tip and coupled with the dilator pusher,
wherein the guide extension hub comprises a clip configured to receive and engage the dilator pusher.

19. The guide extension catheter system of claim 18, wherein the guide extension hub comprises a handle portion proximal of the clip.

20. The guide extension catheter system of claim 18, further comprising:
a carrier tube configured to extend over the clip and the dilator pusher while the clip is engaged with the dilator pusher.
